(19) United States
(12) Patent Application Publication
(10) Pub. No.: US 2024/0355419 A1
(43) Pub. Date: Oct. 24, 2024

(54) RAPID ASSEMBLY OF MULTIPLEX GRNA ARRAYS

(71) Applicant: UT-Battelle, LLC, Oak Ridge, TN (US)

(72) Inventors: Xiaohan Yang, Oak Ridge, TN (US); Md Mahmudul Hassan, Oak Ridge, TN (US); Stanton Martin, Oak Ridge, TN (US); Gerald A. Tuskan, Oak Ridge, TN (US); Guoliang Yuan, Oak Ridge, TN (US)

(21) Appl. No.: 18/640,635

(22) Filed: Apr. 19, 2024

Related U.S. Application Data
(60) Provisional application No. 63/460,723, filed on Apr. 20, 2023.

(51) Int. Cl. G16B 25/20; C12N 9/22; C12N 15/10; C12N 15/11; G16B 35/20

(52) U.S. Cl. CPC G16B 25/20; C12N 9/22; C12N 15/1096; C12N 15/111; G16B 35/20; C12N 2310/121; C12N 2310/123; C12N 2310/20; C12Y 301/26005; C12Y 301/26011

(57) ABSTRACT

The present disclosure is directed to polycistronic guide RNAs, DNA encoding polycistronic gRNA, multiplex CRISPR vectors, a plurality of component DNA fragments for assembly into a DNA encoding a polycistronic gRNA array, a plurality of primer pairs for making a plurality of component DNA fragments to be assembled into a DNA encoding a polycistronic gRNA, and methods of making multiplex CRISPR vectors. The current disclosure is directed to multiplexed CRISPR technologies that have great potential for pathway engineering and genome editing. In the current disclosure describes efficient assembly of tRNA/Csy4/Ribozyme-based gRNA arrays which can be produced in a quick and effective process.

### CROSS REFERENCE TO RELATED APPLICATIONS

This application claims the benefit of U.S. Provisional Patent Application No. 63/460,723, filed Apr. 20, 2023, the contents of which is incorporated herein by reference in its entirety.

### STATEMENT REGARDING FEDERALLY SPONSORED RESEARCH

The United States Government has rights in this invention pursuant to contract no. DE-AC05-00OR22725 between the United States Department of Energy and UT-Battelle, LLC.

### INCORPORATION BY REFERENCE OF SEQUENCE LISTING

The Sequence Listing in an XML file, named as 42885_4991_2_SequenceListing.xml of 16 KB, created on Apr. 18, 2024, and submitted to the United States Patent and Trademark Office via EFS-Web, is incorporated herein by reference.

### BACKGROUND

Multiplexed CRISPR technologies are highly effective DNA editing platforms for multigene editing. The three distinct strategies for multiplexed guide RNA (gRNA) ex-pression are: (1) conventional arrayed multiple, individual gRNA ex-pression cassettes, in which each gRNA is transcribed from a separate RNA polymerase III (Pol III) promoter; (2) CRISPR arrays, in which each gRNA is processed via a native CRISPR processing mechanism; and (3) synthetic gRNA arrays, wherein a single RNA transcript is processed post-transcriptionally into multiple individual gRNAs by RNA-cleaving enzymes. Notably, synthetic gRNA arrays in genome editing have resulted in higher efficacy of gene disruption in yeast, Drosophila and plants. Still, a challenge constraining the use of multiplexed CRISPR is the complicated vector design and construction. Although different approaches have been reported for optimizing multiplex gRNA cloning, a series of intermediate vectors and multistep modular cloning are usually required. However, creating such arrayed architectures remains technically challenging-partially because of the presence of highly repetitive DNA sequences, which prevent multiplexed CRISPR from being widely adopted in various applications.

### SUMMARY

In some aspects, the present disclosure is directed to polycistronic guide RNAs, DNA encoding polycistronic gRNA, multiplex CRISPR vectors, a plurality of component DNA fragments for assembly into a DNA encoding a polycistronic gRNA array, a plurality of primer pairs for making a plurality of component DNA fragments to be assembled into a DNA encoding a polycistronic gRNA, and methods of making multiplex CRISPR vectors.

In one aspect, the present disclosure is directed to a polycistronic guide RNA (gRNA) array, comprising:
nucleotide sequences of a plurality of guide RNAs (gRNAs) for use in a CRISPR-Cas system; wherein:
   a) each nucleotide sequence of a gRNA in the array:
      i) comprises a gRNA targeting sequence and a gRNA binding sequence, wherein the gRNA targeting sequence in each nucleotide sequence of a gRNA is unique to that gRNA; and the gRNA binding sequence is common to all the gRNAs in the array; and
      i) is linked at the 5′ end to a common RNA cleavage recognition sequence (5′ RCRS); and
   b) upon cleavage by an RNA cleaving agent specific for the RNA cleavage recognition sequence, the polycistronic gRNA array generates the plurality of gRNAs.

In some embodiments, each nucleotide sequence of a gRNA in the array is also linked at the 3′ end to a common RNA cleavage recognition sequence (3′ RCRS), wherein the 3′ RCRS is different from the 5′ RCRS. In some embodiments, the CRISPR-Cas system is a CRISPR-Cas9 system or a CRISPR-Cas12 system. In some embodiments, the 5′ RCRS and the 3′ RCRS are selected from a recognition sequence of a ribozyme, a recognition sequence of a tRNA ribonuclease, or a recognition sequence of a Csy4. In some embodiments, the recognition sequence of a ribozyme is the recognition sequence of Hammerhead ribozyme (HH) or the recognition sequence of hepatitis delta virus ribozyme (HDV). In some embodiments, one of the 5′ RCRS and the 3′ RCRS is the recognition sequence of HH, and the other one is the recognition sequence of HDV. In some embodiments, the tRNA ribonucleases are RNase P and RNase Z.

In some embodiments, the CRISPR-Cas system is a CRISPR-Cas9 system, and wherein the 5′ RCRS is selected from a recognition sequence of a ribozyme, a recognition sequence of a tRNA ribonuclease, or a recognition sequence of Csy4.

In some embodiments, the CRISPR-Cas system is a CRISPR-Cas12 system, wherein the nucleotide sequence of each gRNA comprises a LbCpf1 (Cas12a) CRISPR-RNA (crRNA) repeat at the 5′ end, wherein the crRNA repeat is downstream of the 5′ RCRS and upstream of the gRNA targeting sequence; and wherein the crRNA repeat in each nucleotide sequence of a gRNA is common to all the gRNAs in the array. In some embodiments, the 5′ RCRS is the recognition sequence of a first ribozyme; and the 3′ end of the gRNA targeting sequence in each gRNA is linked to a common RCRS (3′ RCRS), wherein the 3′ RCRS comprises the recognition sequence of a second ribozyme; wherein the first and second ribozymes are not the same ribozyme; and wherein upon cleavage by the first and second ribozymes, the polycistronic gRNA array generates the plurality of individual gRNAs. In some embodiments, a) the first ribozyme is Hammerhead ribozyme (HH), and the second ribozyme is hepatitis delta virus ribozyme (HDV); or b) the first ribozyme is HDV and the second ribozyme is HH.

In some embodiments, the array includes at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 gRNAs. In some embodiments, the array includes no more than 20 gRNAs.

In some aspects, the current disclosure is directed to a DNA encoding a polycistronic gRNA described herein.

In some aspects, the current disclosure is directed to a multiplex CRISPR vector, comprising:
a) a DNA encoding a polycistronic gRNA described herein, and
b) a destination vector which comprises, from 5′ to 3′:
   i) a promoter;
   ii) a first recognition sequence of a type IIS restriction enzyme;
   iii) the reverse complement of a second recognition sequence of the type IIS restriction enzyme, and
   iv) a terminator;
      wherein the DNA is integrated into the destination vector between the first recognition sequence and the reverse complement of the second recognition sequence of the type IIS restriction enzyme. In some embodiments, the destination vector further comprises a pol II promoter, a Cas9 sequence and corresponding terminator. In some embodiments, the destination vector further comprises a pol II promoter, a Cas12a sequence and corresponding terminator. In some embodiments, the destination vector further comprises a marker sequence. In some embodiments, the type IIS restriction enzyme is selected from the group of BsaI, AarI, BbsI, BbsI-HF, BsmbI-v2, BspQI, BtgZI, Esp3I, PaqCI and SapI.

Certain aspects of the current disclosure are directed to a plurality of component DNA fragments for assembly into a DNA encoding a polycistronic gRNA array, wherein:
the total number of component DNA fragments is n+1, the total number of gRNAs in the polycistronic gRNA array is n, and n is equal or greater than 2;
the component DNA fragments are designated as the first to the (n+1)^{th} component DNA fragments, and the gRNAs in the polycistronic gRNA array are designated as the first to the nth gRNA;
the DNA encoding a polycistronic gRNA array is generated when the component DNA fragments are assembled in the order of the first to the (n+1)^{th} component DNA fragment in the 5′ to 3′ orientation, wherein:
   a) the first component DNA fragment comprises, from 5′ to 3′:
      i) the recognition sequence of a type IIS restriction enzyme,
      ii) an upstream vector matching overhang sequence of variable length (e.g., 2, 3, 4, 5, or 6-bp),
      iii) a nucleotide sequence encoding a common 5′ RNA cleavage recognition sequence (5′ RCRS),
      iv) a nucleotide sequence encoding a 5′ portion of the targeting sequence of a first gRNA and comprising a downstream overhang sequence unique to the first gRNA; and
      v) the reverse complement of the recognition sequence of the type IIS restriction enzyme;
   b) for each of the second to the (n+1)^{th} component DNA fragments, with “p” representing a number from 2 to n, a p^{th} component DNA fragment comprises, from 5′ to 3′:
      i) the recognition sequence of the type IIS restriction enzyme,
      ii) a nucleotide sequence comprising an upstream overhang sequence and encoding a 3′ portion of the targeting sequence of the (p−1)^{th} gRNA, wherein the upstream overhang sequence is unique to the (p−1)^{th} gRNA and complementary to the downstream (e.g., 2, 3, 4, 5, or 6-bp) overhang sequence in the (p−1)^{th} component DNA fragment;
      iii) a nucleotide sequence encoding a common gRNA binding sequence;
      iv) a nucleotide sequence encoding the common 5′ RCRS,
      v) a nucleotide sequence encoding a 5′ portion of the targeting sequence of the p^{th} gRNA and comprising a downstream overhang sequence unique to the p^{th} gRNA; and
      vi) the reverse complement of the recognition sequence of the type IIS restriction enzyme; and
   c) the (n+1)^{th} component DNA fragment comprises, from 5′ to 3′:
      i) the recognition sequence of the type IIS restriction enzyme;
      ii) a nucleotide sequence comprising an upstream overhang sequence and encoding a 3′ portion of the targeting sequence of the nth gRNA, wherein the upstream overhang sequence is unique to the nth gRNA and complementary to the downstream overhang sequence in the nth component DNA fragment;
      iii) a nucleotide sequence encoding the common gRNA binding sequence;
      iv) a downstream vector matching nucleotide overhang sequence; and
      v) the reverse complement of the recognition sequence of the type IIS restriction enzyme.

In some embodiments, the type IIS restriction enzyme is selected from the group of BsaI, AarI, BbsI, BbsI-HF, BsmbI-v2, BspQI, BtgZI, Esp3I, PaqCI and SapI.

Certain aspects of the current disclosure are directed to a plurality of primer pairs for making a plurality of component DNA fragments to be assembled into a DNA encoding a polycistronic gRNA array, wherein:
the total number of primer pairs is n+1, for making n+1 component DNA fragments to be assembled into a DNA encoding a polycistronic gRNA array for n gRNAs, with n being equal or greater than 2;
the primer pairs are designated as the first to the (n+1)^{th} primer pair, the component DNA fragments are designated as the first to the (n+1)^{th} component DNA fragments, and the gRNAs in the polycistronic gRNA array are designated as the first to the nth gRNA;
the DNA encoding a polycistronic gRNA array is generated when the component DNA fragments are assembled in the order of the first to the (n+1)^{th} component DNA fragment in the 5′ to 3′ orientation, wherein:
   a) the first primer pair comprises a forward primer and a reverse primer, wherein:
   the forward primer of the first primer pair comprises, from 5′ to 3′:
      (i) the recognition sequence of a type IIS restriction enzyme;
      (ii) an upstream vector matching overhang sequence; and
      (iii) a template specific sequence (e.g., a sequence encoding a 5′ portion of an RNA cleavage recognition sequence (RCRS); and
   the reverse primer of the first primer pair comprises, from 5′ to 3′:
      (i) the recognition sequence of the type IIS restriction enzyme;
      (ii) a sequence encoding a 5′ portion of the targeting sequence of the first gRNA and comprising a downstream overhang sequence unique to the first gRNA; and
      (iii) a template specific sequence (e.g., a sequence encoding a 3′ portion of the RNA cleavage recognition sequence (RCRS);
   b) for each of the second to the (n+1)^{th} primer pairs, with “p” representing a number from 2 to n, a p^{th} primer pair comprises a forward primer and a reverse primer, wherein:
   the forward primer of the p^{th} primer pair comprises, from 5′ to 3′:
      (i) the recognition sequence of the type IIS restriction enzyme,
      (ii) a nucleotide sequence comprising an upstream overhang sequence and encoding a 3′ portion of the targeting sequence of the (p−1)^{th} gRNA, wherein the upstream overhang sequence is unique to the (p−1)^{th} gRNA and complementary to the downstream overhang sequence in the reverse primer of the (p−1)^{th} primer pair, and
      (iii) a template specific sequence (e.g., a sequence encoding a 5′ portion of a common gRNA binding sequence); and
   the reverse primer of the p^{th} primer pair comprises, from 5′ to 3′:
      (i) the recognition sequence of the type IIS restriction enzyme;
      (ii) a sequence encoding a 5′ portion of the targeting sequence of the p^{th} gRNA and comprising a downstream overhang sequence unique to the p^{th} gRNA; and
      (iii) a template specific sequence (e.g., a sequence encoding a 3′ portion of an RCRS);
   c) the (n+1)^{th} primer pair comprises a forward primer and a reverse primer, wherein:
   the forward primer the (n+1)^{th} primer pair comprises, from 5′ to 3′:
      (i) the recognition sequence of the type IIS restriction enzyme;
      (ii) a nucleotide sequence comprising an upstream overhang sequence and encoding a 3′ portion of the targeting sequence of the nth gRNA, wherein the upstream overhang sequence is unique to the nth gRNA and complementary to the downstream (4-bp) overhang sequence in the reverse primer of the nth primer pair; and
      (iii) a template specific sequence (e.g., a sequence encoding a 5′ portion of the common gRNA binding sequence); and
   the reverse primer of the (n+1)^{th} primer pair comprises, from 5′ to 3′:
      (i) the recognition sequence of the type IIS restriction enzyme;
      (ii) a downstream vector matching overhang sequence; and
      (iii) a template specific sequence (e.g., a sequence encoding a 3′ portion of the common gRNA binding sequence).

In some embodiments, the length of the overhang sequences (OH) range from 2 nucleotides to 8 nucleotides based on the type IIS restriction enzyme. In some embodiments, the type IIS restriction enzyme recognition site is flanked on the 5′ end by two additional base pairs for enhancing the restriction enzyme digestion of Polymerase Chain Reaction products. In some embodiments, the type IIS restriction enzyme is BsaI and the overhangs are 4 nucleotides in length.

In some aspects, the disclosure is directed to a method of making a multiplex CRISPR vector described herein, the method comprising:
a) selecting an organism and gRNA mode;
b) inputting a gRNA list and destination vector sequences into a database; wherein the gRNA list comprises nucleotide sequences of a plurality of gRNAs, wherein each nucleotide sequence of a gRNA in the array comprises a gRNA targeting sequence and a gRNA binding sequence;
c) optimizing nucleotide overhangs from gRNA sequences, comprising;
   i) identifying candidate overhangs from each of the gRNA sequences;
   ii) identifying all overhang combinations with a pairwise crossmatch score of less than 30 from identified candidate overhangs in step (c) (i); and
   iii) identifying the best overhang combination with the highest total self-match score for assembling the gRNA array;
d) designing primer pairs;
e) generating component DNA fragments by combining the corresponding forward primer (F[n]), predefined template sequence, and reverse primer PCR amplification, wherein:
   i) n+1 component DNA fragments are to be assembled into a DNA encoding a polycistronic gRNA array for n gRNAs, with n being equal or greater than 2;
   ii) the component DNA fragments are designated as the first to the (n+1)^{th} component DNA fragments, and the gRNAs in the polycistronic gRNA array are designated as the first to the nth gRNA; and
   iii) the DNA encoding a polycistronic gRNA array is generated when the component DNA fragments are assembled in the order of the first to the (n+1)^{th} component DNA fragment in the 5′ to 3′ orientation;
f) assembling the gRNA array sequence by combining individual component DNA fragments from step (e); and
g) generating assembled vector sequences by connecting user-provided destination vector and assembled gRNA array sequence from step (f). In some embodiments, the method further comprises step (h), downloading text files of all described outputs, including the required oligos (d), component DNA fragments (e), assembled gRNA array sequence (f) and assembled vector sequences (g).

In some embodiments, step (a) of selecting an organism and gRNA mode comprises selecting the type of multi-gRNA expression system, the ligation action, the appropriate restriction enzyme, and the organism type. In some embodiments, step (c) (iii) identifying the best overhang combination with the highest total self-match score for assembling the gRNA array further comprises using an algorithm to choose OH combinations. In some embodiments, step (d) designing primer pairs comprises:
i) identifying candidate primer pairs; wherein:
   the total number of primer pairs is n+1, for making n+1 component DNA fragments to be assembled into a DNA encoding a polycistronic gRNA array for n gRNAs, with n being equal or greater than 2;
   the primer pairs are designated as the first to the (n+1)^{th} primer pair, the component DNA fragments are designated as the first to the (n+1)^{th} component DNA fragments, and the gRNAs in the polycistronic gRNA array are designated as the first to the nth gRNA;
   the DNA encoding a polycistronic gRNA array is generated when the component DNA fragments are assembled in the order of the first to the (n+1)^{th} component DNA fragment in the 5′ to 3′ orientation;
wherein:
a) the first primer pair comprises:
a forward primer comprising, from 5′ to 3′:
   (i) the recognition sequence of a type IIS restriction enzyme;
   (ii) an upstream vector matching overhang sequence; and
   (iii) a template specific sequence (e.g., a sequence encoding a 5′ portion of an RNA cleavage recognition sequence (RCRS)); and
a reverse primer comprising, from 5′ to 3′:
   (i) the recognition sequence of the type IIS restriction enzyme;
   (ii) a sequence encoding a 5′ portion of the targeting sequence of the first gRNA and comprising a downstream overhang sequence unique to the first gRNA; and
   (iii) a template specific sequence (e.g., a sequence encoding a 3′ portion of an RCRS);
b) for each of the second to the (n+1)^{th} primer pairs, with “p” representing a number from 2 to n, a p^{th} primer pair comprises:
a forward primer comprising, from 5′ to 3′:
   (i) the recognition sequence of the type IIS restriction enzyme;
   (ii) a nucleotide sequence comprising an upstream overhang sequence and encoding a 3′ portion of the targeting sequence of the (p−1)^{th} gRNA, wherein the upstream overhang sequence is unique to the (p−1)^{th} gRNA and complementary to the downstream overhang sequence in the reverse primer of the (p−1)^{th} primer pair; and
   (iii) a template specific sequence (e.g., a sequence encoding a 5′ portion of a common gRNA binding sequence); and
a reverse primer comprising, from 5′ to 3′:
   (i) the recognition sequence of the type IIS restriction enzyme;
   (ii) a sequence encoding a 5′ portion of the targeting sequence of the p^{th} gRNA and comprising a downstream overhang sequence unique to the p^{th} gRNA; and
   (iii) a template specific sequence (e.g., a sequence encoding a 3′ portion of an RCRS);
c) the (n+1)^{th} primer pair comprises:
a forward primer comprising, from 5′ to 3′:
   (i) the recognition sequence of the type IIS restriction enzyme;
   (ii) a nucleotide sequence comprising an upstream overhang sequence and encoding a 3′ portion of the targeting sequence of the nth gRNA, wherein the upstream overhang sequence is unique to the nth gRNA and complementary to the downstream overhang sequence in the reverse primer of the nth primer pair; and
   (iii) a template specific sequence (e.g., a sequence encoding a 5′ portion of the common gRNA binding); and
a reverse primer comprising, from 5′ to 3′:
   (i) the recognition sequence of the type IIS restriction enzyme;
   (ii) a downstream vector matching overhang sequence; and
   (iii) a template specific sequence (e.g., a sequence encoding a 3′ portion of the common gRNA binding).

In some embodiments, the assembly of the component DNA fragments in step (e) are assembled in the order of the first to the (n+1)^{th} component DNA fragment in the 5′ to 3′ orientation, further wherein:
a) the first component DNA fragment comprises, from 5′ to 3′:
   i) the recognition sequence of a type IIS restriction enzyme;
   ii) an upstream vector matching overhang sequence;
   iii) a nucleotide sequence encoding a (self-cleaving) ribonuclease;
   iv) a nucleotide sequence encoding a 5′ portion of the targeting sequence of a first gRNA and comprising a downstream overhang sequence unique to the first gRNA; and
   v) the reverse complement of the recognition sequence of the type IIS restriction enzyme;
b) for each of the second to the (n+1)^{th} component DNA fragments, with “p” representing a number from 2 to n, a p^{th} component DNA fragment comprises, from 5′ to 3′:
   i) the recognition sequence of the type IIS restriction enzyme;
   ii) a nucleotide sequence comprising an upstream overhang sequence and encoding a 3′ portion of the targeting sequence of the (p−1)^{th} gRNA, wherein the upstream overhang sequence is unique to the (p−1)^{th} gRNA and complementary to the downstream overhang sequence in the (p−1)^{th} component DNA fragment;
   iii) a nucleotide sequence encoding a common gRNA binding sequence;
   iv) a nucleotide sequence encoding a common RNA cleavage recognition sequence (RCRS);
   v) a nucleotide sequence encoding a 5′ portion of the targeting sequence of the p^{th} gRNA and comprising a downstream overhang sequence unique to the p^{th} gRNA; and
   vi) the reverse complement of the recognition sequence of the type IIS restriction enzyme; and
c) the (n+1)^{th} component DNA fragment comprises, from 5′ to 3′:
   i) the recognition sequence of the type IIS restriction enzyme;
   ii) a nucleotide sequence comprising an upstream overhang sequence and encoding a 3′ portion of the targeting sequence of the nth gRNA, wherein the upstream overhang sequence is unique to the nth gRNA and complementary to the downstream overhang sequence in the nth component DNA fragment;
   iii) a nucleotide sequence encoding a common gRNA binding sequence;
   iv) a downstream vector matching nucleotide overhang sequence; and
   v) the reverse complement of the recognition sequence of the type IIS restriction enzyme.

In some embodiments of the disclosure, step (f) assembling the gRNA array sequence further comprises combining the individual component DNA fragments from step (e) wherein the assembled gRNA array sequence comprises:
nucleotide sequences of a plurality of gRNAs;
wherein each nucleotide sequence of a gRNA in the array:
   comprises a gRNA targeting sequence and a gRNA binding sequence, wherein the gRNA targeting sequence in each nucleotide sequence of a gRNA is unique to that gRNA, and the gRNA binding sequence is common to all the gRNAs in the array; and
   is linked at the 5′ end to a common RNA cleavage recognition sequence (5′ RCRS); and
wherein upon cleavage by the RNA ribonuclease, the polycistronic gRNA array generates the plurality of gRNAs.

In some embodiments, step (g) generating assembled vector sequences further comprises performing Golden Gate assembly with the gRNA and a CRISPR vector. In some embodiments, the method of making the multiplex CRISPR vector further comprises step (i) cloning the vector. In some embodiments, the length of the overhang sequences ranges from 2 nucleotides to 8 nucleotides based on the type IIS restriction enzyme. In some embodiments, the type IIS restriction enzyme is BsaI and the length of the overhang sequences is 4.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1A-D. A one-step cloning system for multiplex guide RNA expression. (A) The Golden Gate assembly for preparing a plant binary vector expressing multiple gRNAs under a Pol III promoter. Each tRNA-gRNA unit is amplified from a predesigned template vector. All tRNA-gRNA parts are ligated into a plant binary vector through Golden Gate assembly. (B) An example of a polycistronic gRNA. (C) Design of primers used for amplifying gRNA-tRNA parts. [n+1] fragments are required for assembly [n] gRNAs. Purple sequences are added to enhance BsaI digestion of PCR products. Blue sequences indicate the BsaI sites. The NNNN sequences are gRNA spacer with length ranging from 4 bp to 20 bp. The red NNNN indicates the distinct 4-bp overhangs of one embodiment of the disclosure required for the ligation of two DNA parts after digestion with BsaI during golden gate assembly. Underlined sequences are specific for template sequences. Included are the following identified sequences and SEQ ID NOs: FP [1] TAGGTCTCCGGTCAACAAAGCACCAGT (SEQ ID NO: 1); RP [1] ATGGTCTCANNNNNNNNTGCACCAGCCGGGAATCG (SEQ ID NO: 2); FP [n] TAGGTCTCCNNNNNNNNGTTTTAGAGCTAGAAATAGCAAG (SEQ ID NO: 3); RP [n] ATGGTCTCANNNNNNNNTGCACCAGCCGGAATCG (SEQ ID NO: 4); FP [n+1] TAGGTCTCCNNNNNNNNGTTTTAGAGCTAGAAATAGCAAG (SEQ ID NO: 5); RP [n+1] ATGGTCTCACCACTTTTTCAAGTTGATAACGG (SEQ ID NO: 6); (D) Cloning procedures of multiplex gRNAs.

FIG. 2A-E. Rapid and highly efficient assembly of the gRNA array. (A) The cloning efficiencies of plant tRNA-gRNA system with different number of gRNAs using the PARA method. Error bars represent standard deviations (SD) of three replicates. (B) Structure of four widely used constructs for expressing gRNA array in CRISPR-Cas based multiplexed genome editing. (1) tRNA, pre-tRNAGly gene varying in different organisms; (2) Csy4, 20-bp Csy4 hairpin; RZ, (3) 15-bp ribozyme cleavage site; (4) HH-HDV-RB, HH hammerhead Ribozyme and HDV hepatitis delta virus ribozyme. Constructs 1, 2, and 3 are in the form of an embodiment of the disclosure having a structure when viewed in linear form from 5′ to 3′ of a primer (arrow)-tRNA-gRNA₁-tRNA-gRNA₂-tRNA-gRNAₙ-termination sequence (example given for construct 1). Construct 4 is an example of embodiments having a structure in the linear form from 5 to 3′ of a primer (arrow)-HH-gRNA₁-HDV-HH-gRNA₂-HDV-HH-gRNAₙ-termination sequence. (C) The cloning efficiencies of four different gRNA array systems harbor eight gRNAs using PARA method. Error bars represent SD of three replicates. (D) The features of the PARAweb designed for the assembly of gRNA arrays. (E) The screenshot of the interface of PARAweb.

FIG. 3A-B. The workflow of PARAweb design. (A) The steps on the user front-end. (B) The steps on the server side.

FIG. 4A-D. Global optimization of OHs from 8 gRNA sequences. (A) Identification of candidate OHs from each of the 20-nt gRNA sequences. A gRNA sequence 20 bases in length has 17 four-nucleotide substrings (20−4+1). (B) Identification of all OH combinations with a pairwise crossmatch score<30 from identified candidate OHs in panel (A). (C) Identification of the best OH combination with the highest total self-match score for assembling the gRNA array. OH1 is predefined based on backbone sequence (GGTC) while OH10 is predefined on backbone sequence (GTGG). (D) The location of each OH in the gRNA array.

FIG. 5A-B. Valid Cas9 vector that is compatible with the PARA method. (A) The valid vector must and can only contain two BsaI recognition GGTCTC(1/5)\hat{ } sites between the PoIII/III promoter and the corresponding terminator. (B) Represents an example of one embodiment of a valid destination (CRISPR) vector, in which there is a predefined window sequence flanked by the promoter and the terminator of a gRNA-array transcription unit. The promoter-window sequence-terminator of upper strand is:
AGGGAGCACCATTGGTCGGAGACCAACGGTCTCGGTGGCACCGAGTCGG TGCTTTTTTTTCCCTTTCCT (SEQ ID NO: 7) while the promoter-window sequence-terminator of upper strand is:
TCCCTCGTGGTAACCAGCCTCTGGRRGCCAGAGCCACCGTGGCTCAGCCA CGAAAAAAAAGGGAAAGGA (SEQ ID NO: 8). As one example of one embodiment of a predefined window sequence used to code PARAweb, the upper strand of the window sequence is
GGTCGGAGACCAACGGTCTCGGTGGCACCGAGTCGGTGCTTTTTTT (SEQ ID NO: 9) while the lower strand of the window sequence is
CCAGCCTCTGGRRGCCAGAGCCACCGTGGCTCAGCCACGAAAAAAA (SEQ ID NO: 10). To export an assembled vector sequence using PARAweb, the given vector should contain the indicated window sequence.

FIG. 6A-B. Structure of template vectors for PCR-amplification of component DNA fragments. (A) Template vector type 1. (B) Template vector type 2.

FIG. 7A-F. Colony plates for assembly of different number of single guide RNAs (sgRNA) in different expression system. (A) Colony plates for assembly of different number of sgRNAs in plant tRNA expression system using PCR products. Loading volume varies. (B) Colony plates for assembly of eight sgRNAs in Ribozyme expression system using PCR products. Loading volume=300 μL. (C) Colony plates for assembly of eight sgRNAs in Csy4 expression system using PCR products. Loading volume=900 μL. (D) Colony plates for assembly of eight sgRNAs in HDV-HH-RB expression system using PCR products. Loading volume=300 μL. (E) Colony plates for assembly of eight sgRNAs in HDV-HH-RB expression system using gBlocks. Loading volume=900 μL. (F) Colony plates for assembly of eight gRNAs in plant tRNA expression system using PARA-based PCR products. Loading volume=900 μL. The total volume of outgrowth medium is usually 900 μL.

FIG. 8A-I. Colony PCR for the screening of transformants for plant tRNA expression system. (A) Colony PCR of two gRNAs assembly. (B) Colony PCR of four gRNAs assembly. (C) Colony PCR of six gRNAs assembly. (D) Colony PCR of eight gRNAs assembly. (E) Colony PCR of ten gRNAs assembly. (F) Colony PCR of 12 gRNAs assembly. (G) Colony PCR of 14 gRNAs assembly. (H) Colony PCR of 16 gRNAs assembly. (I) Colony PCR of 18 gRNAs assembly.

### DETAILED DESCRIPTION

The current disclosure relates to solutions which address the current limitations regarding arrayed architectures, specifically the presence of highly repetitive DNA sequences, which prevent multiplexed CRISPR from being widely adopted in various applications. To address these limitations, we developed the prime assembly of gRNA arrays (PARA) method for the fast cloning of multiple gRNAs in an array into a CRISPR vector via a one-pot reaction in a microcentrifuge tube. The disclosed method provides for fast, efficient, one-step construction of diverse gRNA arrays to facilitate multiplexed genome editing and gene regulation in a wide range of organisms. Furthermore, disclosed herein is a webtool, termed “PARAweb,” for optimal design of high-fidelity overhangs from a list of gRNA sequences. PARAweb displays ready-to-use primers for PCR-amplification of component fragments, along with simulation of cloning steps. As a flexible, universal, and all-inclusive methodology for joining gRNA arrays, PARA is dedicated to accelerating the development and application of multiplexed CRISPR in agriculture, medicine, and bioenergy in the future.

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. As used herein, the term “comprises” means “includes.” Thus, “comprising a nucleic acid molecule” means “including a nucleic acid molecule” without excluding other elements. It is further to be understood that any and all base sizes given for nucleic acids are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. All references, including patent applications and patents, are herein incorporated by reference in their entireties.

As used herein, the term “complementary” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “CRISPR” stands for “Clustered Regularly Interspaced Short Palindromic Repeats”. The CRISPR RNA array is a defining feature of CRISPR systems. The term “CRISPR” refers to the architecture of the array which includes constant direct repeats (DRs) interspaced with the variable spacers. Engineered CRISPR systems contain two components: a guide RNA and a CRISPR-associated endonuclease (Cas protein). The gRNA is a short synthetic RNA composed of a scaffold sequence necessary for Cas-binding and a user-defined ˜20 nucleotide spacer that defines the genomic target to be modified, i.e. a specific RNA sequence that recognizes the region of interest in the target DNA. Thus, one can change the genomic target of the Cas protein by simply changing the target sequence present in the gRNA.

The three distinct strategies for multiplexed guide RNA (gRNA) expression are: (1) conventional arrayed multiple, individual gRNA (gRNA) expression cassettes, in which each gRNA is transcribed from a separate RNA polymerase III (Pol III) promoter; (2) CRISPR arrays, in which each gRNA is processed via a native CRISPR processing mechanism; and (3) synthetic gRNA arrays, wherein a single RNA transcript is processed post-transcriptionally into multiple individual gRNAs by RNA-cleaving enzymes. As used herein, “gRNA array” refers to a combination of independently expressing gRNAs organized in a linear fashion. As used herein, the term “polycistronic” refers to the encoding of two or more separate proteins encoded on a single molecule of RNA. In some embodiments, the polycistronic gRNA arrays of the disclosure comprise up to 20 gRNA.

As used herein, the term “encoding” refers to the specific sequences of nucleotides in a polynucleotide, such as a gene, a cDNA, or an mRNA, acting as templates for the synthesis of other polymers and macromolecules in biological processes having either a defined sequence of nucleotides (i.e., rRNA, tRNA and mRNA) or a defined sequence of amino acids. For example, a gene will encode a protein if transcription and translation of the mRNA corresponding to that gene produces the protein in a cell or other biological system. The nucleotide sequence is identical to the mRNA sequence is termed the “coding strand”. The nucleotide sequence used as the template for transcription of a gene or cDNA is termed the “non-coding strand.” Both the coding and the non-coding strands can be referred to as encoding the protein or other product of that gene or cDNA.

The term “spacer sequence” refers to a spacer sequence of a gRNA of a CRISPR Cas system, as is known in the art. The guide RNA spacer sequence is complementary to a corresponding target nucleic acid sequence, referred to in the art as a “protospacer”. The term spacer sequence is understood by those of skill in the art and may include any polynucleotide having sufficient complementarity with a target nucleic acid sequence (i.e. “protospacer”) to hybridize with the target nucleic acid sequence and direct sequence-specific binding of a CRISPR complex to the target sequence. A CRISPR complex may include the guide RNA and a Cas protein, such as a Cas9 or Cas12 protein.

As used herein, the term “restriction endonuclease recognition site” or “cut site” is intended to include, but is not limited to, a particular nucleic acid sequence to which one or more restriction enzymes bind, resulting in cleavage of a DNA molecule either at the restriction endonuclease recognition sequence itself, or at a sequence distal to the restriction endonuclease recognition sequence. Restriction enzymes include, but are not limited to, type I enzymes, type II enzymes, type IIS enzymes, type III enzymes and type IV enzymes. Additional exemplary enzymes include programmable nucleases such as Cas9, TALEN and ZFN as is known to those of skill in the art. The REBASE database provides a comprehensive database of information about restriction enzymes, DNA methyltransferases and related proteins involved in restriction-modification. It contains both published and unpublished work with information about restriction endonuclease recognition sites and restriction endonuclease cleavage sites, isoschizomers, commercial availability, crystal and sequence data (see Roberts et al. (2005) Nucl. Acids Res. 33: D230, incorporated herein by reference in its entirety for all purposes).

In certain aspects, primers of the present invention include one or more restriction endonuclease recognition sites that enable type IIS enzymes to cleave the nucleic acid several base pairs 3′ to the restriction endonuclease recognition sequence. As used herein, the term “type IIS” refers to a restriction enzyme that cuts at a site remote from its recognition sequence. Type IIS enzymes are known to cut at a known distance from their recognition sites ranging from 0 to 20 base pairs. Examples of Type IIs endonucleases include, but are not limited to, enzymes that produce a 3′ overhang, such as, for example, Bsr I, Bsm I, BstF5 I, BsrD I, Bts I, Mnl I, BciV I, Hph I, Mbo II, Eci I, Acu I, Bpm I, Mme I, BsaX I, Bcg I, Bae I, Bfi I, TspDT I, TspGW I, Taq II, Eco57 I, Eco57M I, Gsu I, Ppi I, and Psr I; enzymes that produce a 5′ overhang such as, for example, BsmA I, Ple I, Fau I, Sap I, BspM I, SfaN I, Hga I, Bvb I, Fok I, BceA I, BsmF I, Ksp632 I, Eco31 I, Esp3 I, Aar I; and enzymes that produce a blunt end, such as, for example, Mly I and Btr I. Type-IIs endonucleases are commercially available and are well known in the art (New England Biolabs, Beverly, Mass.). Information about the recognition sites, cut sites and conditions for digestion using type IIs endonucleases may be found, for example, on the Worldwide web at neb.com/nebecomm/enzymefindersearch bytypeIIs.asp). Restriction endonuclease sequences and restriction enzymes are well known in the art and restriction enzymes are commercially available (New England Biolabs, Ipswich, Mass.). Exemplary restriction enzymes include BtgZI, BsaI, sapI, aarl, and BsmBI and the like. One of skill will be readily able to identify other useful restriction enzymes from public information such as websites and periodicals based on the present disclosure such that an exhaustive list need not be presented here. In some embodiments, the restriction enzyme used is the same at the 5′ and 3′ ends of the nucleotide.

According to certain aspects, the restriction endonuclease cut site may be within an oligonucleotide and may be introduced during in situ synthesis. According to one aspect, the inner restriction endonuclease cut sites separating spacer sequences may be different from each other. This design feature allows one to select a particular restriction endonuclease to cut between two desired spacer sequences. As the cutting produces free ends of the nucleic acid, a desired nucleic acid sequence can be inserted into the cut site, i.e., between the two ends created by the restriction endonuclease cutting the nucleic acid, using methods known to those of skill in the art, such as ligation.

As used herein, “vector” refers to nucleic acid molecule into which a foreign nucleic acid molecule can be introduced without disrupting the ability of the vector to replicate and/or integrate in a host cell. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art.

A vector can include nucleic acid sequences that permit it to replicate in a host cell, such as an origin of replication. A vector can also include one or more selectable marker genes and other genetic elements known in the art. An integrating vector is capable of integrating itself into a host nucleic acid. An expression vector is a vector that contains the necessary regulatory sequences to allow transcription and translation of inserted gene or genes.

One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. In some embodiments, the vector is a lentivirus (such as an integration-deficient lentiviral vector) or adeno-associated viral (AAV) vector.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

Certain vectors are capable of directing the expression of genes to which they are operatively-linked. Such vectors are referred to herein as “expression vectors.” Common expression vectors are often in the form of plasmids. Recombinant expression vectors can comprise a nucleic acid provided herein (such as a guide RNA [which can be expressed from an RNA sequence or a RNA sequence], nucleic acid encoding a Cas protein, i.e. Cas9 or Cas12) in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Regulatory elements are contemplated for use with the methods and constructs described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector may comprise one or more pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6, 7SK and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter and Pol II promoters described herein. Also encompassed by the term “regulatory element” are enhancer elements, such as WPRE; CMV enhancers; the R-U5′ segment in LTR of HTLV-I (Mol. Cell. Biol., Vol. 8 (1), p. 466-472, 1988); SV40 enhancer; and the intron sequence between exons 2 and 3 of rabbit β-globin (Proc. Natl. Acad. Sci. USA., Vol. 78 (3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Aspects of the methods described herein may make use of terminator sequences. A terminator sequence includes a section of nucleic acid sequence that marks the end of a gene or operon in genomic DNA during transcription. This sequence mediates transcriptional termination by providing signals in the newly synthesized mRNA that trigger processes which release the mRNA from the transcriptional complex. These processes include the direct interaction of the mRNA secondary structure with the complex and/or the indirect activities of recruited termination factors. Release of the transcriptional complex frees RNA polymerase and related transcriptional machinery to begin transcription of new mRNAs. Terminator sequences include those known in the art.

Polycistronic Guide RNA (gRNA) Array

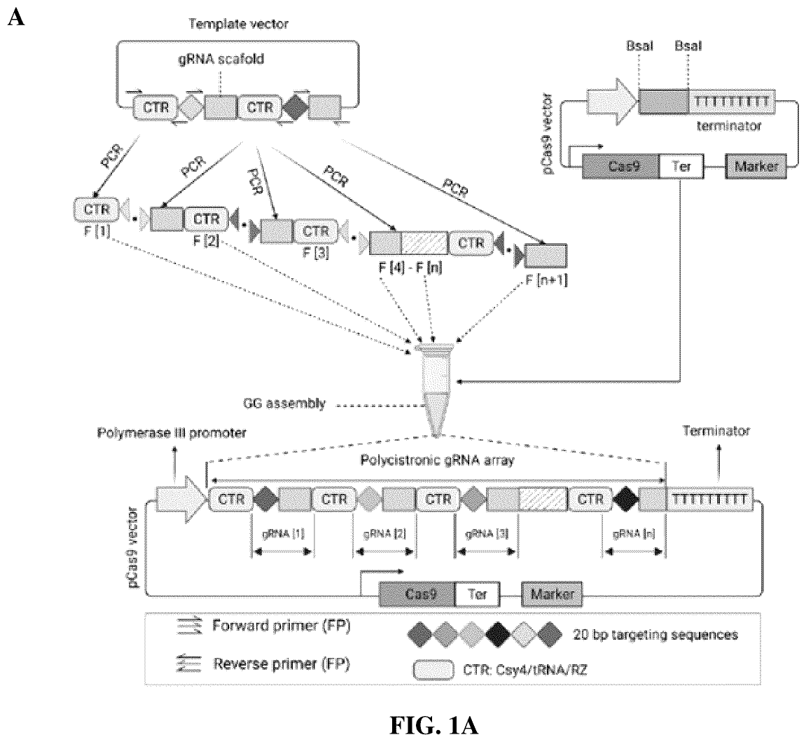

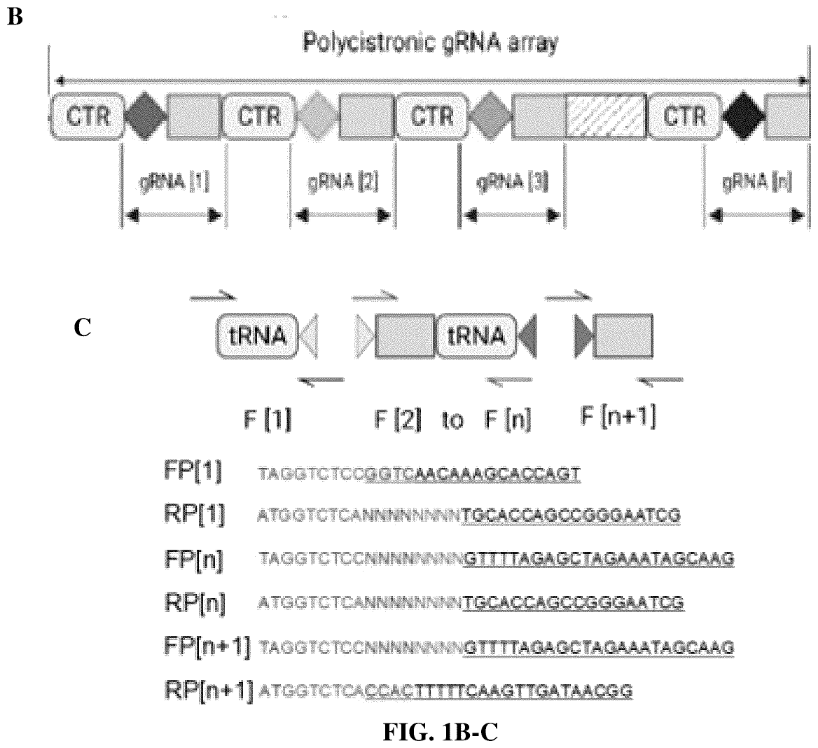

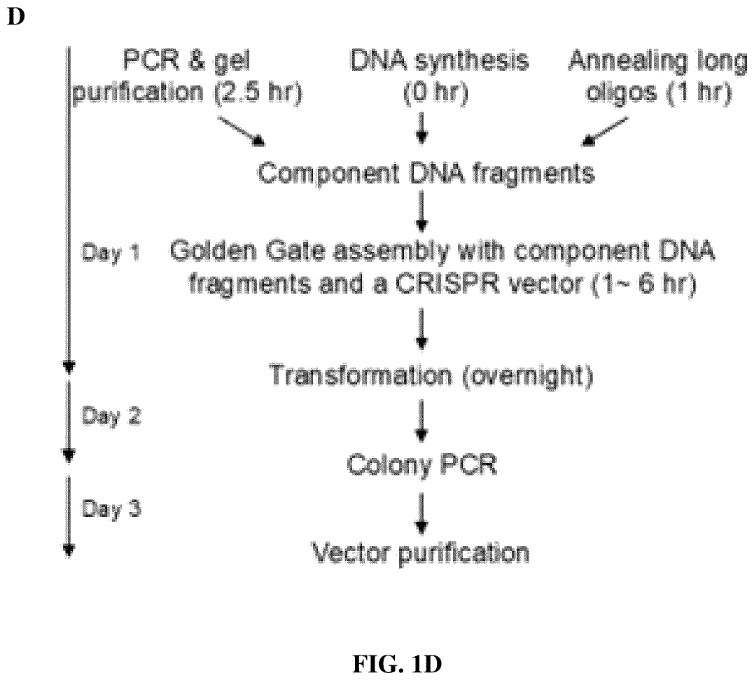

In one aspect, the present disclosure is directed to a polycistronic guide RNA (gRNA) array. An example of a polycistronic gRNA is shown in FIG. 1B while the polycistronic gRNA of FIG. 1B is shown placed in a vector in FIG. 1A. The polycistronic gRNA of some embodiments comprises nucleotide sequences of a plurality of guide RNAs (gRNAs) for use in a CRISPR-Cas system. In some embodiments, each nucleotide sequence of an gRNA in the array comprises a gRNA targeting sequence and a gRNA binding sequence, wherein the gRNA targeting sequence in each nucleotide sequence of a gRNA is unique to that gRNA and the gRNA binding sequence is common to all the gRNAs in the array. Additionally, in some embodiments, each nucleotide sequence of a gRNA in the array is linked at the 5′ end to a common RNA cleavage recognition sequence (5′ RCRS). In some embodiments, the 5′RCRS is a recognition sequence of a ribozyme, a recognition sequence of a tRNA ribonuclease, or a recognition sequence of a Csy4. In some embodiments, the recognition sequence of a ribozyme is the recognition sequence of Hammerhead ribozyme (HH) or the recognition sequence of hepatitis delta virus ribozyme (HDV). In some embodiments, the tRNA ribonucleases are RNase P and RNase Z.

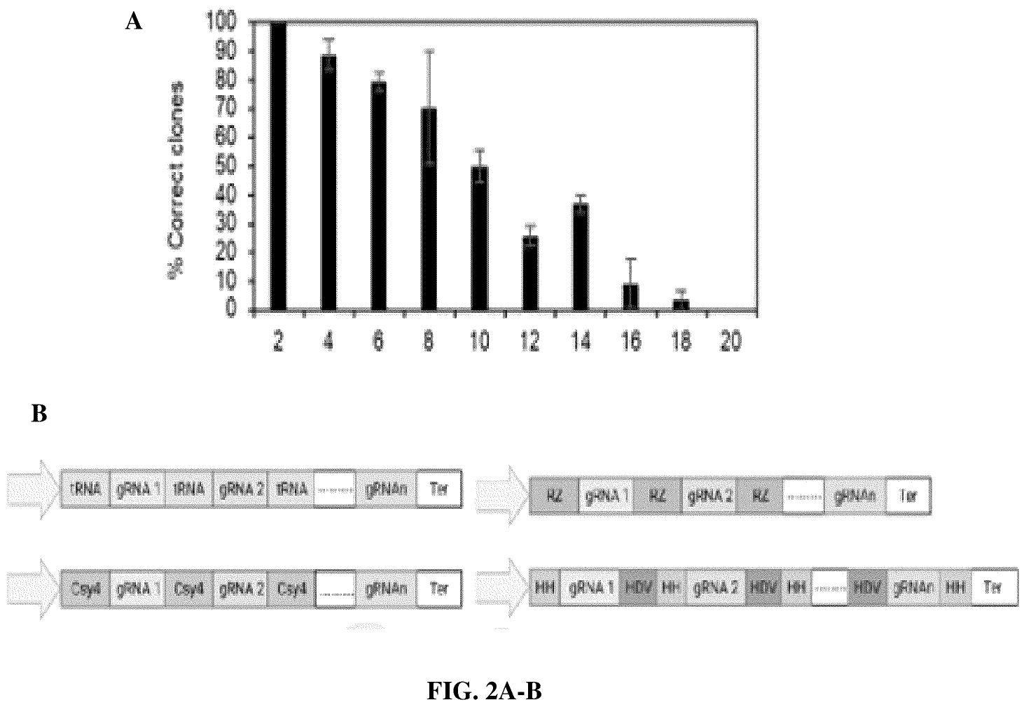

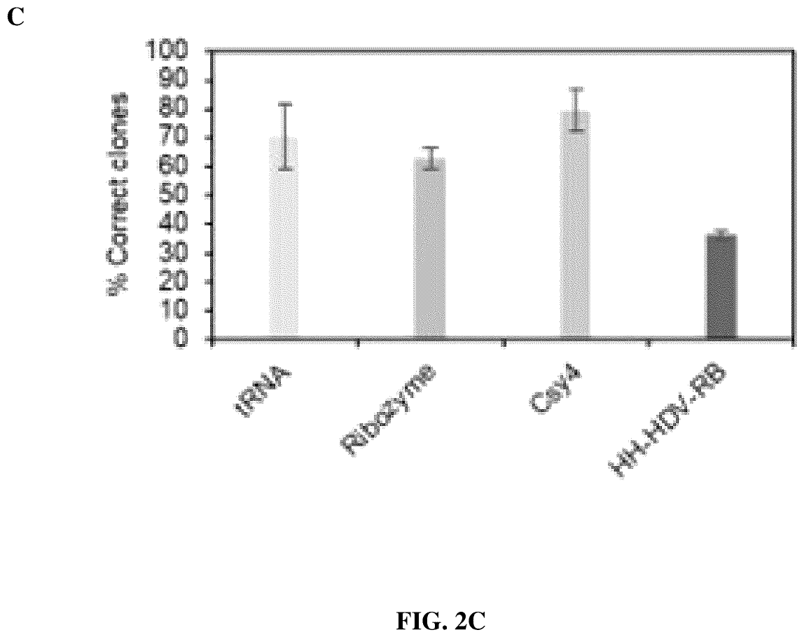

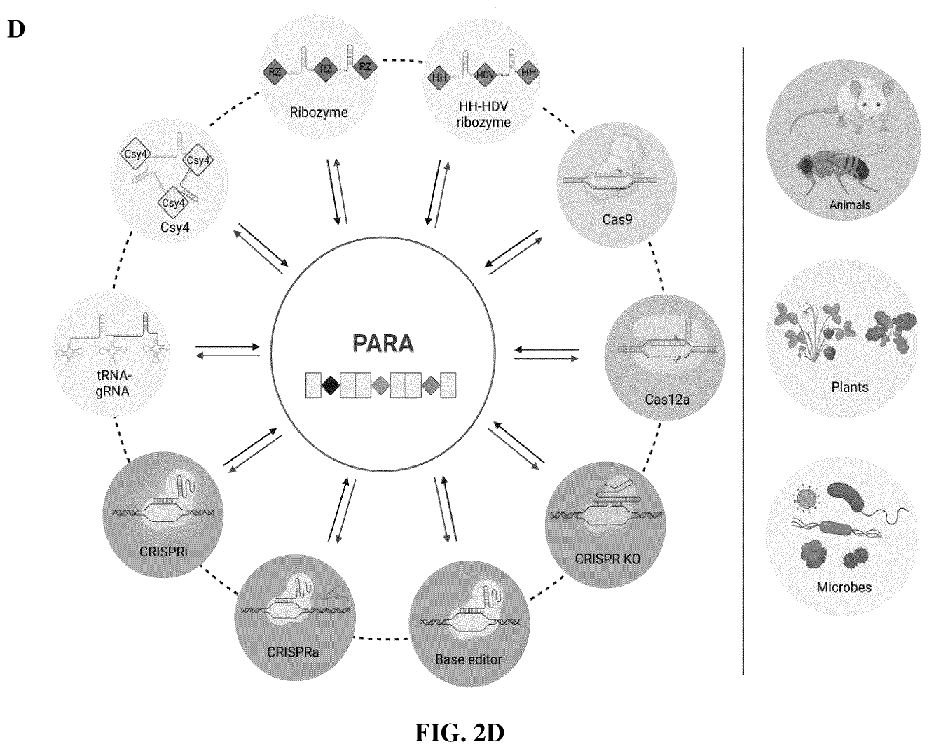

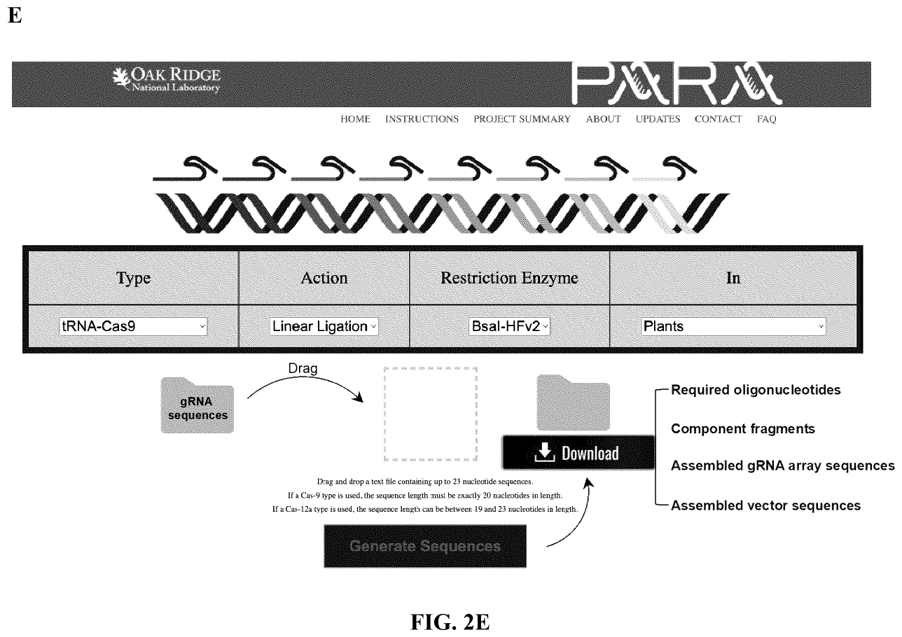

In such embodiments, when viewing the polycistronic gRNA array in a linear form, there is a 5′RCRS linked to a first gRNA followed by a 5′RCRS linked to a second gRNA followed by a 5′RCRS linked to a third gRNA, etc. An example of this can be seen in FIG. 2B where in linear form from 5′ to 3′ there is a primer (arrow)-tRNA-gRNA₁-tRNA-gRNA₂-tRNA-gRNAₙ-termination sequence. In effect, each middle gRNA is flanked by an RNA cleavage recognition sequence (RCRS) but there is one RCRS separating the gRNAs. This is due to there not being a need to duplicate the RCRS. Such a linear sequence allows for the RNA cleaving agent specific for the RCRS to separate each gRNA individually. In other words, the RCRS between the two gRNA is, in a sense, shared by the two gRNA. In some embodiments, upon cleavage by an RNA cleaving agent specific for the RNA cleavage recognition sequence, the polycistronic gRNA array generates the plurality of gRNAs.

In some embodiments, each nucleotide sequence of a gRNA in the array is also linked at the 3′ end to a common RNA cleavage recognition sequence (3′ RCRS), wherein the 3′ RCRS is different from the 5′ RCRS. In some embodiments, the 5′ RCRS and the 3′ RCRS are selected from a recognition sequence of a ribozyme, a recognition sequence of a tRNA ribonuclease, or a recognition sequence of a Csy4. In some embodiments, the recognition sequence of a ribozyme is the recognition sequence of HH or the recognition sequence of HDV. In some embodiments, one of the 5′ RCRS and the 3′ RCRS is the recognition sequence of HH, and the other one is the recognition sequence of HDV. In some embodiments, the tRNA ribonucleases are RNase P and RNase Z.

Unlike embodiments where gRNA of the array are linked to only 5′ RCRS, in embodiments where the gRNA of the array are also linked to an RCRS at the 3′ end, there are two RCRS in between each gRNA of the array. In such embodiments, when viewing the polycistronic gRNA array in a linear form, there is a 5′RCRS linked to a first gRNA linked to a 3′ RCRS followed by a 5′RCRS linked to a second gRNA linked to a 3′ RCRS followed by a 5′RCRS linked to a third gRNA linked to a 3′ RCRS, etc. An example can be seen in FIG. 2B where in linear form from 5 to 3′ there is a primer (arrow)-HH-gRNA₁-HDV-HH-gRNA₂-HDV-HH-gRNAₙ-termination sequence. In this example, the HH is the 5′ RCRS and the HDV is the 3′ RCRS.

In some embodiments, the CRISPR-Cas system is a CRISPR-Cas9 system. In some embodiments, the 5′ RCRS is selected from a recognition sequence of a ribozyme, a recognition sequence of a tRNA ribonuclease, or a recognition sequence of Csy4. In some embodiments, the recognition sequence of a ribozyme is the recognition sequence of HH or the recognition sequence of HDV. In some embodiments, the tRNA ribonucleases are RNase P and RNase Z.

In some embodiments, the CRISPR-Cas system is a CRISPR-Cas12 system, wherein the nucleotide sequence of each gRNA comprises a CRISPR-RNA (crRNA) repeat at the 5′ end, wherein the crRNA repeat is downstream of the 5′ RCRS and upstream of the gRNA targeting sequence; and wherein the crRNA repeat in each nucleotide sequence of a gRNA is common to all the gRNAs in the array. In some embodiments, the crRNA repeat is a LbCpf1 (Cas12a) repeat. In some embodiments, the 5′ RCRS is the recognition sequence of a first ribozyme; and the 3′ end of the gRNA targeting sequence in each gRNA is linked to a common RCRS (3′ RCRS), wherein the 3′ RCRS comprises the recognition sequence of a second ribozyme; wherein the first and second ribozymes are not the same ribozyme; and wherein upon cleavage by the first and second ribozymes, the polycistronic gRNA array generates the plurality of individual gRNAs. In some embodiments, the first ribozyme is HH, and the second ribozyme is HDV. In some embodiments, the first ribozyme is HDV and the second ribozyme is HH.

In some embodiments, the array includes at least 2 gRNAs. In some embodiments, the array includes at least 3 gRNAs. In some embodiments, the array includes at least 4 gRNAs. In some embodiments, the array includes at least 5 gRNAs. In some embodiments, the array includes at least 6 gRNAs. In some embodiments, the array includes at least 7 gRNAs. In some embodiments, the array includes at least 8 gRNAs. In some embodiments, the array includes at least 9 gRNAs. In some embodiments, the array includes at least 10 gRNAs. In some embodiments, the array includes at least 11 gRNAs. In some embodiments, the array includes at least 12 gRNAs. In some embodiments, the array includes at least 13 gRNAs. In some embodiments, the array includes at least 14 gRNAs. In some embodiments, the array includes at least 15 gRNAs. In some embodiments, the array includes at least 16 gRNAs. In some embodiments, the array includes at least 17 gRNAs. In some embodiments, the array includes at least 18 gRNAs. In some embodiments, the array includes at least 19 gRNAs. In some embodiments, the array includes no more than 20 gRNAs.

### DNA and Multiplex CRISPR Vector

In some aspects, the current disclosure is directed to a DNA encoding a polycistronic gRNA described herein. In some embodiments, the DNA encodes a polycistronic gRNA comprising nucleotide sequences of a plurality of gRNAs for use in a CRISPR-Cas system. In some embodiments, the DNA encodes a polycistronic gRNA wherein each nucleotide sequence of an gRNA in the array comprises a gRNA targeting sequence and a gRNA binding sequence, wherein the gRNA targeting sequence in each nucleotide sequence of a gRNA is unique to that gRNA and the gRNA binding sequence is common to all the gRNAs in the array. In some embodiments, the DNA encodes a polycistronic gRNA wherein each nucleotide sequence of a gRNA in the array is linked at the 5′ end to a common 5′ RCRS. In some embodiments, the DNA encodes a polycistronic gRNA wherein the 5′RCRS is a recognition sequence of a ribozyme, a recognition sequence of a tRNA ribonuclease, or a recognition sequence of a Csy4. In some embodiments, the DNA encodes a polycistronic gRNA wherein the recognition sequence of a ribozyme is the recognition sequence of HH or the recognition sequence of HDV. In some embodiments, the DNA encodes a polycistronic gRNA wherein the tRNA ribonucleases are RNase P and RNase Z.

In some embodiments, the DNA encodes a polycistronic gRNA wherein each nucleotide sequence of a gRNA in the array is also linked at the 3′ end to a common 3′ RCRS, wherein the 3′ RCRS is different from the 5′ RCRS. In some embodiments, the DNA encodes a polycistronic gRNA wherein the 5′ RCRS and the 3′ RCRS are selected from a recognition sequence of a ribozyme, a recognition sequence of a tRNA ribonuclease, or a recognition sequence of a Csy4. In some embodiments, the DNA encodes a polycistronic gRNA wherein the recognition sequence of a ribozyme is the recognition sequence of HH or the recognition sequence of HDV. In some embodiments, the DNA encodes a polycistronic gRNA wherein one of the 5′ RCRS and the 3′ RCRS is the recognition sequence of HH, and the other one is the recognition sequence of HDV. In some embodiments, the DNA encodes a polycistronic gRNA wherein the tRNA ribonucleases are RNase P and RNase Z.

In some embodiments, the DNA encodes a polycistronic gRNA wherein the polycistronic gRNA array includes at least 2 gRNAs. In some embodiments, the DNA encodes a polycistronic gRNA wherein the polycistronic gRNA array includes at least 3 gRNAs. In some embodiments, the DNA encodes a polycistronic gRNA wherein the polycistronic gRNA array includes at least 4 gRNAs. In some embodiments, the DNA encodes a polycistronic gRNA wherein the polycistronic gRNA array includes at least 5 gRNAs. In some embodiments, the DNA encodes a polycistronic gRNA wherein the polycistronic gRNA array includes at least 6 gRNAs. In some embodiments, the DNA encodes a polycistronic gRNA wherein the polycistronic gRNA array includes at least 7 gRNAs. In some embodiments, the DNA encodes a polycistronic gRNA wherein the polycistronic gRNA array includes at least 8 gRNAs. In some embodiments, the DNA encodes a polycistronic gRNA wherein the polycistronic gRNA array includes at least 9 gRNAs. In some embodiments, the DNA encodes a polycistronic gRNA wherein the polycistronic gRNA array includes at least 10 gRNAs. In some embodiments, the DNA encodes a polycistronic gRNA wherein the polycistronic gRNA array includes at least 11 gRNAs. In some embodiments, the DNA encodes a polycistronic gRNA wherein the polycistronic gRNA array includes at least 12 gRNAs. In some embodiments, the DNA encodes a polycistronic gRNA wherein the polycistronic gRNA array includes at least 13 gRNAs. In some embodiments, the DNA encodes a polycistronic gRNA wherein the polycistronic gRNA array includes at least 14 gRNAs. In some embodiments, the DNA encodes a polycistronic gRNA wherein the polycistronic gRNA array includes at least 15 gRNAs. In some embodiments, the DNA encodes a polycistronic gRNA wherein the polycistronic gRNA array includes at least 16 gRNAs. In some embodiments, the DNA encodes a polycistronic gRNA wherein the polycistronic gRNA array includes at least 17 gRNAs. In some embodiments, the DNA encodes a polycistronic gRNA wherein the polycistronic gRNA array includes at least 18 gRNAs. In some embodiments, the DNA encodes a polycistronic gRNA wherein the polycistronic gRNA array includes at least 19 gRNAs. In some embodiments, the DNA encodes a polycistronic gRNA wherein the polycistronic gRNA array includes no more than 20 gRNAs.

As described herein, the term “encoding” refers to the specific sequences of nucleotides in a polynucleotide, such as a gene, a cDNA, or an mRNA, acting as templates for the synthesis of other polymers and macromolecules in biological processes having either a defined sequence of nucleotides (i.e., rRNA, tRNA and mRNA) or a defined sequence of amino acids. For example, a gene will encode a protein if transcription and translation of the mRNA corresponding to that gene produces the protein in a cell or other biological system. The nucleotide sequence is identical to the mRNA sequence is termed the “coding strand”. The nucleotide sequence used as the template for transcription of a gene or cDNA is termed the “non-coding strand.” Both the coding and the non-coding strands can be referred to as encoding the protein or other product of that gene or cDNA.

In some aspects, the current disclosure is directed to a multiplex CRISPR vector, comprising:
a) a DNA encoding a polycistronic gRNA described herein, and
b) a destination vector which comprises, from 5′ to 3′:
   i) a promoter;
   ii) a first recognition sequence of a type IIS restriction enzyme;
   iii) the reverse complement of a second recognition sequence of the type IIS restriction enzyme, and
   iv) a terminator;
      wherein the DNA is integrated into the destination vector between the first recognition sequence and the reverse complement of the second recognition sequence of the type IIS restriction enzyme.

In some embodiments, the destination vector further comprises a Cas9 sequence and corresponding terminator. In some embodiments, the destination vector further comprises a Cas12a sequence and corresponding terminator. In some embodiments, the destination vector further comprises a marker sequence. In some embodiments, the type IIS restriction enzyme is BsaI AarI, BbsI, BbsI-HF, BsmbI-v2, BspQI, BtgZI, Esp3I, PaqCI and SapI

As described supra, a “vector” refers to nucleic acid molecule into which a foreign nucleic acid molecule can be introduced without disrupting the ability of the vector to replicate and/or integrate in a host cell. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art.

A vector can include nucleic acid sequences that permit it to replicate in a host cell, such as an origin of replication. A vector can also include one or more selectable marker genes and other genetic elements known in the art. An integrating vector is capable of integrating itself into a host nucleic acid. An expression vector is a vector that contains the necessary regulatory sequences to allow transcription and translation of inserted gene or genes.

One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. In some embodiments, the vector is a lentivirus (such as an integration-deficient lentiviral vector) or adeno-associated viral (AAV) vector.

### Component DNA Fragments

Certain aspects of the current disclosure are directed to a plurality of component DNA fragments for assembly into a DNA encoding a polycistronic gRNA array, wherein:
the total number of component DNA fragments is n+1, the total number of gRNAs in the polycistronic gRNA array is n, and n is equal or greater than 2;
the component DNA fragments are designated as the first DNA component fragment to the (n+1)^{th} component DNA fragment, and the gRNAs in the polycistronic gRNA array are designated as the first to the n^{th} gRNA;
the DNA encoding a polycistronic gRNA array is generated when the component DNA fragments are assembled in the order of the first to the (n+1)^{th} component DNA fragment in the 5′ to 3′ orientation, wherein:
   a) the first component DNA fragment comprises, from 5′ to 3′:
      i) the recognition sequence of a type IIS restriction enzyme,
      ii) an upstream vector matching overhang sequence of variable length (e.g., 2, 3, 4, 5, or 6-bp),
      iii) a nucleotide sequence encoding a common 5′ RNA cleavage recognition sequence (5′ RCRS),
      iv) a nucleotide sequence encoding a 5′ portion of the targeting sequence of a first gRNA and comprising a downstream overhang sequence unique to the first gRNA; and
      v) the reverse complement of the recognition sequence of the type IIS restriction enzyme;
b) for each of the second to the (n+1)^{th} component DNA fragments, with “p” representing a number from 2 to n, a p^{th} component DNA fragment comprises, from 5′ to 3′:
   i) the recognition sequence of the type IIS restriction enzyme,
   ii) a nucleotide sequence comprising an upstream overhang sequence and encoding a 3′ portion of the targeting sequence of the (p−1)^{th} gRNA, wherein the upstream overhang sequence is unique to the (p−1)^{th} gRNA and complementary to the downstream (e.g., 2, 3, 4, 5, or 6-bp) overhang sequence in the (p−1)^{th} component DNA fragment;
   iii) a nucleotide sequence encoding a common gRNA binding sequence;
   iv) a nucleotide sequence encoding the common 5′ RCRS,
   v) a nucleotide sequence encoding a 5′ portion of the targeting sequence of the p^{th} gRNA and comprising a downstream overhang sequence unique to the p^{th} gRNA; and
   vi) the reverse complement of the recognition sequence of the type IIS restriction enzyme; and
c) the (n+1)^{th} component DNA fragment comprises, from 5′ to 3′:
   i) the recognition sequence of the type IIS restriction enzyme;
   ii) a nucleotide sequence comprising an upstream overhang sequence and encoding a 3′ portion of the targeting sequence of the nth gRNA, wherein the upstream overhang sequence is unique to the nth gRNA and complementary to the downstream overhang sequence in the nth component DNA fragment;
   iii) a nucleotide sequence encoding the common gRNA binding sequence;
   iv) a downstream vector matching nucleotide overhang sequence; and
   v) the reverse complement of the recognition sequence of the type IIS restriction enzyme.

In some embodiments, the type IIS restriction enzyme is, FokI, BsrI, BsmI, BstF5I, BsrDI, BtsI, MnlI, BciVI, HphI, MboII, EciI, Acul, BpmI, Mmel, BsaXI, BcgI, BaeI, BfiI, TspDTI, TspGWI, TaqII, Eco57I, Eco57MI, GsuI, PpiI, PsrI; BsmAI, PleI, FauI, SapI, BspMI, SfaNI, HgaI, BvbI, BceAI, BsmFI, Ksp632I, Eco31I, Esp3I, or Aar I.

As an example of the DNA component fragments, an array with 4 gRNA would have a first DNA component fragment (F[1]), a second DNA component fragment (F[2]), a third DNA component fragment (F[3]=F[p−1]), a fourth DNA component fragment (F[4]=F[n]), and a fifth (and last) DNA component fragment (F[5]=F[n+1]). This is represented in FIG. 1A.

In some embodiments, n equals 2. In some embodiments, n equals 3. In some embodiments, n equals 4. In some embodiments, n equals 5. In some embodiments, n equals 6. In some embodiments, n equals 7. In some embodiments, n equals 8. In some embodiments, n equals 9. In some embodiments, n equals 10. In some embodiments, n equals 11. In some embodiments, n equals 12. In some embodiments, n equals 13. In some embodiments, n equals 14. In some embodiments, n equals 15. In some embodiments, n equals 16. In some embodiments, n equals 17. In some embodiments, n equals 18. In some embodiments, n equals 19. In some embodiments, n equals 20.

### Primer Pairs

Certain aspects of the current disclosure are directed to a plurality of primer pairs for making a plurality of component DNA fragments to be assembled into a DNA encoding a polycistronic gRNA array, wherein:
the total number of primer pairs is n+1, for making n+1 component DNA fragments to be assembled into a DNA encoding a polycistronic gRNA array for n gRNAs, with n being equal or greater than 2;
the primer pairs are designated as the first to the (n+1)^{th} primer pair, the component DNA fragments are designated as the first to the (n+1)^{th} component DNA fragments, and the gRNAs in the polycistronic gRNA array are designated as the first to the nth gRNA;
the DNA encoding a polycistronic gRNA array is generated when the component DNA fragments are assembled in the order of the first to the (n+1)^{th} component DNA fragment in the 5′ to 3′ orientation, wherein:
   a) the first primer pair comprises a forward primer and a reverse primer, wherein:
   the forward primer of the first primer pair comprises, from 5′ to 3′:
      (i) the recognition sequence of a type IIS restriction enzyme;
      (ii) an upstream vector matching overhang sequence; and
      (iii) a template specific sequence (e.g., a sequence encoding a 5′ portion of an RNA cleavage recognition sequence (RCRS); and
   the reverse primer of the first primer pair comprises, from 5′ to 3′:
      (i) the recognition sequence of the type IIS restriction enzyme;
      (ii) a sequence encoding a 5′ portion of the targeting sequence of the first gRNA and comprising a downstream overhang sequence unique to the first gRNA; and
      (iii) a template specific sequence (e.g., a sequence encoding a 3′ portion of the RNA cleavage recognition sequence (RCRS);
   b) for each of the second to the (n+1)^{th} primer pairs, with “p” representing a number from 2 to n, a p^{th} primer pair comprises a forward primer and a reverse primer, wherein:
   the forward primer of the p^{th} primer pair comprises, from 5′ to 3′:
      (i) the recognition sequence of the type IIS restriction enzyme,
      (ii) a nucleotide sequence comprising an upstream overhang sequence and encoding a 3′ portion of the targeting sequence of the (p−1)^{th} gRNA, wherein the upstream overhang sequence is unique to the (p−1)^{th} gRNA and complementary to the downstream overhang sequence in the reverse primer of the (p−1)^{th} primer pair, and
      (iii) a template specific sequence (e.g., a sequence encoding a 5′ portion of a common gRNA binding sequence); and
   the reverse primer of the p^{th} primer pair comprises, from 5′ to 3′:
      (i) the recognition sequence of the type IIS restriction enzyme;
      (ii) a sequence encoding a 5′ portion of the targeting sequence of the p^{th} gRNA and comprising a downstream overhang sequence unique to the p^{th} gRNA; and
      (iii) a template specific sequence (e.g., a sequence encoding a 3′ portion of an RCRS);
   c) the (n+1)^{th} primer pair comprises a forward primer and a reverse primer, wherein:
   the forward primer the (n+1)^{th} primer pair comprises, from 5′ to 3′:
      (i) the recognition sequence of the type IIS restriction enzyme;
      (ii) a nucleotide sequence comprising an upstream overhang sequence and encoding a 3′ portion of the targeting sequence of the nth gRNA, wherein the upstream overhang sequence is unique to the nth gRNA and complementary to the downstream overhang sequence in the reverse primer of the nth primer pair; and
      (iii) a template specific sequence (e.g., a sequence encoding a 5′ portion of the common gRNA binding sequence); and
   the reverse primer of the (n+1)^{th} primer pair comprises, from 5′ to 3′:
      (i) the recognition sequence of the type IIS restriction enzyme;
      (ii) a downstream vector matching overhang sequence; and
      (iii) a template specific sequence (e.g., a sequence encoding a 3′ portion of the common gRNA binding sequence).

Using the same array with 4 gRNA from the example above, the an array would have a first primer pair (FP [1] and RP [1]), a second primer pair (FP [2] and RP [2]), a third primer pair (FP [3] and RP [3]=FP [p−1] and RP [p−1]), a fourth primer pair (FP [4] and RP [4]=FP [n] and RP [n]), and a fifth (and last) primer pair (FP [5] and RP [5]=FP [n+1] and RP [n+1]). This is represented in FIG. 1C.

In some embodiments, the length of the overhang sequences (OH) range from 2 nucleotides to 8 nucleotides based on the type IIS restriction enzyme. In some embodiments, the length of the OH sequences are 2 nucleotides. In some embodiments, the length of the OH sequences range from 3 nucleotides. In some embodiments, the length of the OH sequences are 4 nucleotides. In some embodiments, the length of the OH sequences range from 5 nucleotides. In some embodiments, the length of the OH sequences are 6 nucleotides. In some embodiments, the length of the OH sequences range from 7 nucleotides. In some embodiments, the length of the OH sequences are 8 nucleotides. The length of the overhang created by type IIS restriction enzymes are known in the art.

In some embodiments, the type IIS restriction enzyme recognition site is flanked on the 5′ end by two additional base pairs for enhancing the restriction enzyme digestion of Polymerase Chain Reaction products. An example of this can be seen in FIG. 1C. In some embodiments, the type IIS restriction enzyme is BsaI and the overhangs are 4 nucleotides in length.

### Methods of Making Multiplex CRISPR Vector

In some aspects, the disclosure is directed to a method of making a multiplex CRISPR vector described herein, the method comprising:
a) selecting an organism and gRNA mode;
b) inputting a gRNA list and destination vector sequences into a database;
   wherein the gRNA list comprises nucleotide sequences of a plurality of gRNAs, wherein each nucleotide sequence of a gRNA in the array comprises a gRNA targeting sequence and a gRNA binding sequence;
c) optimizing nucleotide overhangs from gRNA sequences, comprising;
   i) identifying candidate overhangs from each of the gRNA sequences;
   ii) identifying all overhang combinations with a pairwise crossmatch score of less than 30 from identified candidate overhangs in step (c) (i); and
   iii) identifying the best overhang combination with the highest total self-match score for assembling the gRNA array;
d) designing primer pairs;
e) generating component DNA fragments by combining the corresponding forward primer (F[n]), predefined template sequence, and reverse primer PCR amplification, wherein:
   i) n+1 component DNA fragments are to be assembled into a DNA encoding a polycistronic gRNA array for n gRNAs, with n being equal or greater than 2;
      ii) the component DNA fragments are designated as the first to the (n+1)^{th} component DNA fragments, and the gRNAs in the polycistronic gRNA array are designated as the first to the nth gRNA; and
   iii) the DNA encoding a polycistronic gRNA array is generated when the component DNA fragments are assembled in the order of the first to the (n+1)^{th} component DNA fragment in the 5′ to 3′ orientation;
f) assembling the gRNA array sequence by combining individual component DNA fragments from step (e); and
g) generating assembled vector sequences by connecting user-provided destination vector and assembled gRNA array sequence from step (f). In some embodiments, the method further comprises step (h), downloading text files of all described outputs, including the required oligos (d), component DNA fragments (e), assembled gRNA array sequence (f) and assembled vector sequences (g).

The crossmatch score is determined based on assembly fidelity of the overhang pairs in assembly reactions with BsaI-HFv2 and T4 DNA ligase. Determination of assembly fidelity is presented in https://doi.org/10.1371/journal.pone.0238592, which is herein incorporated by reference in its entirety.

In some embodiments, step (a) of selecting an organism and gRNA mode comprises selecting the type of multi-gRNA expression system, the ligation action, the appropriate restriction enzyme, and the organism type. In some embodiments, step (c) (iii) identifying the best overhang combination with the highest total self-match score for assembling the gRNA array further comprises using an algorithm to choose OH combinations. In some embodiments, step (d) designing primer pairs comprises:
i) identifying candidate primer pairs; wherein:
   the total number of primer pairs is n+1, for making n+1 component DNA fragments to be assembled into a DNA encoding a polycistronic gRNA array for n gRNAs, with n being equal or greater than 2;
   the primer pairs are designated as the first to the (n+1)^{th} primer pair, the component DNA fragments are designated as the first to the (n+1)^{th} component DNA fragments, and the gRNAs in the polycistronic gRNA array are designated as the first to the nth gRNA;
   the DNA encoding a polycistronic gRNA array is generated when the component DNA fragments are assembled in the order of the first to the (n+1)^{th} component DNA fragment in the 5′ to 3′ orientation;
wherein:
a) the first primer pair comprises:
a forward primer comprising, from 5′ to 3′:
   (i) the recognition sequence of a type IIS restriction enzyme;
   (ii) an upstream vector matching overhang sequence; and
   (iii) a template specific sequence (e.g., a sequence encoding a 5′ portion of an RNA cleavage recognition sequence (RCRS)); and
a reverse primer comprising, from 5′ to 3′:
   (i) the recognition sequence of the type IIS restriction enzyme;
   (ii) a sequence encoding a 5′ portion of the targeting sequence of the first gRNA and comprising a downstream overhang sequence unique to the first gRNA; and
   (iii) a template specific sequence (e.g., a sequence encoding a 3′ portion of an RCRS);
b) for each of the second to the (n+1)^{th} primer pairs, with “p” representing a number from 2 to n, a p^{th} primer pair comprises:
a forward primer comprising, from 5′ to 3′:
   (i) the recognition sequence of the type IIS restriction enzyme;
   (ii) a nucleotide sequence comprising an upstream overhang sequence and encoding a 3′ portion of the targeting sequence of the (p−1)^{th} gRNA, wherein the upstream overhang sequence is unique to the (p−1)^{th} gRNA and complementary to the downstream overhang sequence in the reverse primer of the (p−1)^{th} primer pair; and
   (iii) a template specific sequence (e.g., a sequence encoding a 5′ portion of a common gRNA binding sequence); and
a reverse primer comprising, from 5′ to 3′:
   (i) the recognition sequence of the type IIS restriction enzyme;
   (ii) a sequence encoding a 5′ portion of the targeting sequence of the p^{th} gRNA and comprising a downstream overhang sequence unique to the p^{th} gRNA; and
   (iii) a template specific sequence (e.g., a sequence encoding a 3′ portion of an RCRS);
c) the (n+1)^{th} primer pair comprises:
a forward primer comprising, from 5′ to 3′:
   (i) the recognition sequence of the type IIS restriction enzyme;
   (ii) a nucleotide sequence comprising an upstream overhang sequence and encoding a 3′ portion of the targeting sequence of the nth gRNA, wherein the upstream overhang sequence is unique to the nth gRNA and complementary to the downstream overhang sequence in the reverse primer of the nth primer pair; and
   (iii) a template specific sequence (e.g., a sequence encoding a 5′ portion of the common gRNA binding); and
a reverse primer comprising, from 5′ to 3′:
   (i) the recognition sequence of the type IIS restriction enzyme;
   (ii) a downstream vector matching overhang sequence; and
   (iii) a template specific sequence (e.g., a sequence encoding a 3′ portion of the common gRNA binding).

In some embodiments, the assembly of the component DNA fragments in step (e) are assembled in the order of the first to the (n+1)^{th} component DNA fragment in the 5′ to 3′ orientation, further wherein:
a) the first component DNA fragment comprises, from 5′ to 3′:
   i) the recognition sequence of a type IIS restriction enzyme;
   ii) an upstream vector matching overhang sequence;
   iii) a nucleotide sequence encoding a (self-cleaving) ribonuclease;
   iv) a nucleotide sequence encoding a 5′ portion of the targeting sequence of a first gRNA and comprising a downstream overhang sequence unique to the first gRNA; and
   v) the reverse complement of the recognition sequence of the type IIS restriction enzyme;
b) for each of the second to the (n+1)^{th} component DNA fragments, with “p” representing a number from 2 to n, a p^{th} component DNA fragment comprises, from 5′ to 3′:
   i) the recognition sequence of the type IIS restriction enzyme;
   ii) a nucleotide sequence comprising an upstream overhang sequence and encoding a 3′ portion of the targeting sequence of the (p−1)^{th} gRNA, wherein the upstream overhang sequence is unique to the (p−1)^{th} gRNA and complementary to the downstream overhang sequence in the (p−1)^{th} component DNA fragment;
iii) a nucleotide sequence encoding a common gRNA binding sequence;
iv) a nucleotide sequence encoding a common RNA cleavage recognition sequence (RCRS);
v) a nucleotide sequence encoding a 5′ portion of the targeting sequence of the p^{th} gRNA and comprising a downstream overhang sequence unique to the p^{th} gRNA; and
vi) the reverse complement of the recognition sequence of the type IIS restriction enzyme; and
c) the (n+1)^{th} component DNA fragment comprises, from 5′ to 3′:

i) the recognition sequence of the type IIS restriction enzyme;
ii) a nucleotide sequence comprising an upstream overhang sequence and encoding a 3′ portion of the targeting sequence of the nth gRNA, wherein the upstream overhang sequence is unique to the nth gRNA and complementary to the downstream overhang sequence in the nth component DNA fragment;
iii) a nucleotide sequence encoding a common gRNA binding sequence;
iv) a downstream vector matching nucleotide overhang sequence; and
v) the reverse complement of the recognition sequence of the type IIS restriction enzyme.

In some embodiments of the disclosure, step (f) assembling the gRNA array sequence further comprises combining the individual component DNA fragments from step (e) wherein the assembled gRNA array sequence comprises:
nucleotide sequences of a plurality of gRNAs;
wherein each nucleotide sequence of a gRNA in the array:
   comprises a gRNA targeting sequence and a gRNA binding sequence, wherein the gRNA targeting sequence in each nucleotide sequence of a gRNA is unique to that gRNA, and the gRNA binding sequence is common to all the gRNAs in the array; and
   is linked at the 5′ end to a common RNA cleavage recognition sequence (5′ RCRS); and
wherein upon cleavage by the RNA ribonuclease, the polycistronic gRNA array generates the plurality of gRNAs.

In some embodiments, step (g) generating assembled vector sequences further comprises performing Golden Gate assembly with the gRNA and a CRISPR vector. In some embodiments, the method of making the multiplex CRISPR vector further comprises step (i) cloning the vector. In some embodiments, the length of the overhang sequences ranges from 2 nucleotides to 8 nucleotides based on the type IIS restriction enzyme. In some embodiments, the length of the OH sequences are 2 nucleotides. In some embodiments, the length of the OH sequences range from 3 nucleotides. In some embodiments, the length of the OH sequences are 4 nucleotides. In some embodiments, the length of the OH sequences range from 5 nucleotides. In some embodiments, the length of the OH sequences are 6 nucleotides. In some embodiments, the length of the OH sequences range from 7 nucleotides. In some embodiments, the length of the OH sequences are 8 nucleotides. In some embodiments, the type IIS restriction enzyme is BsaI and the length of the overhang sequences is 4. The length of the overhang created by type IIS restriction enzymes are known in the art.

### PARAweb

In one aspect, the technologies described herein provide a Prime Assembly of gRNA Arrays (PARA) method for fast cloning of multiple gRNAs in an array into a CRISPR vector with a single one-pot reaction.

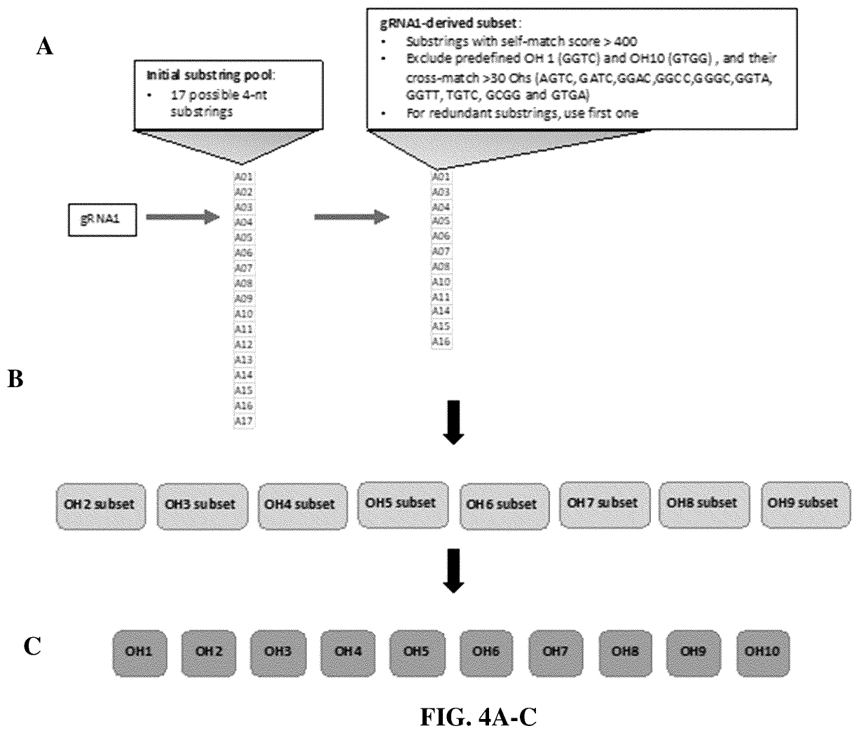

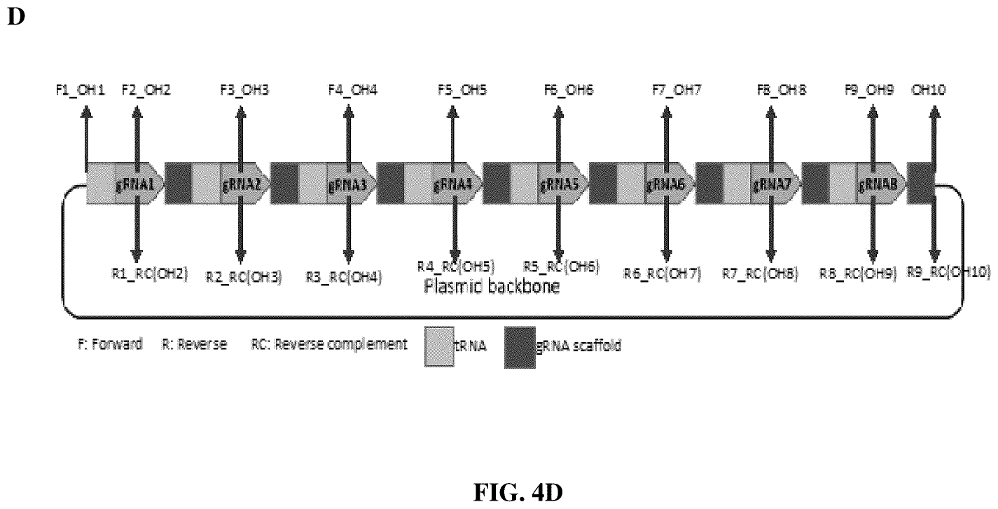

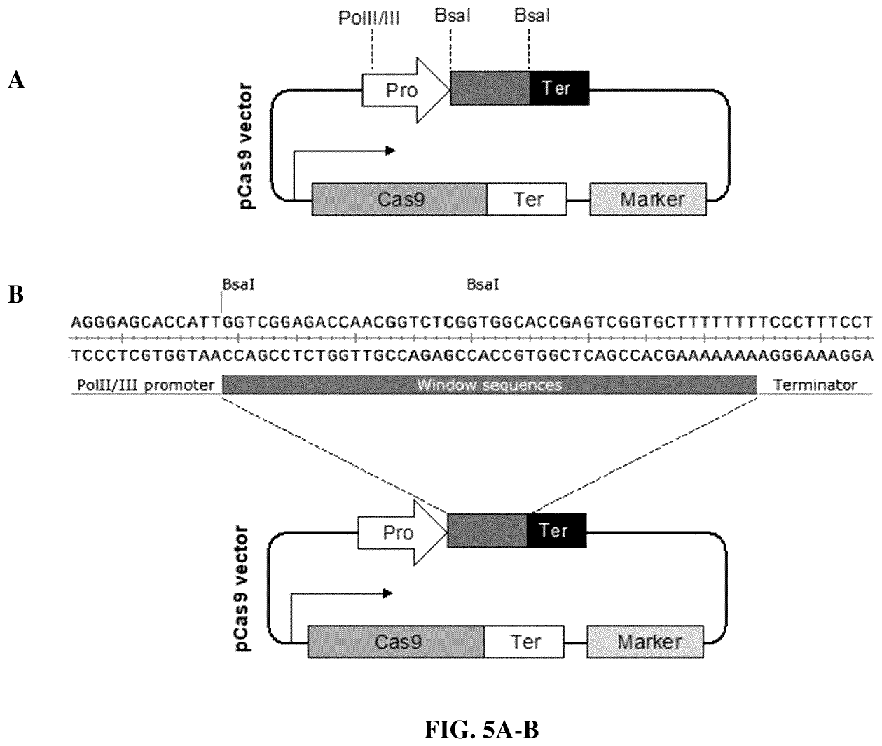

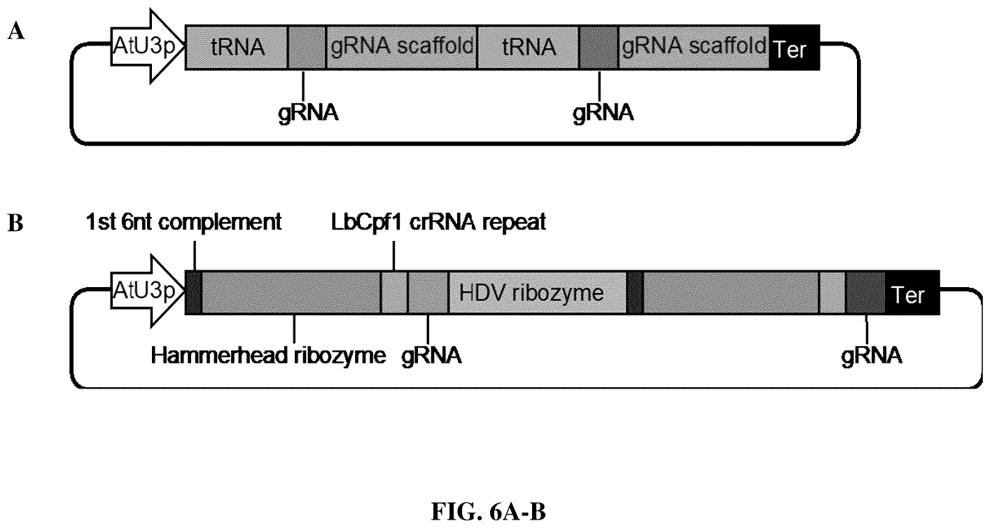

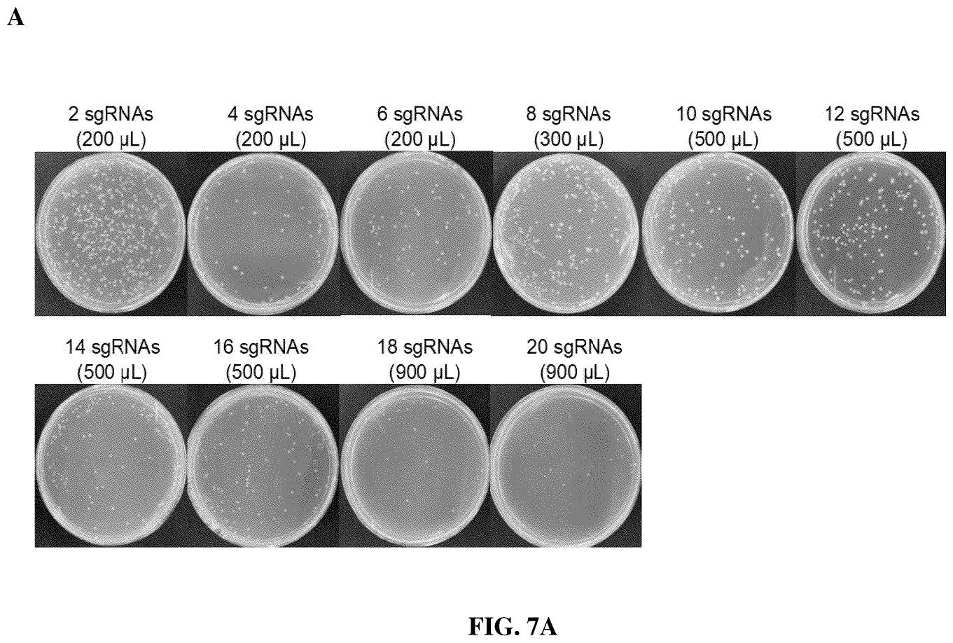

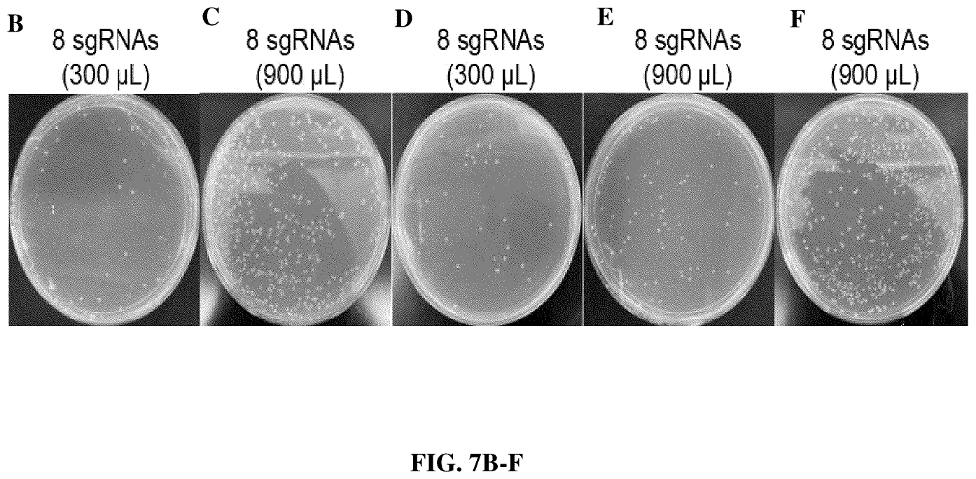

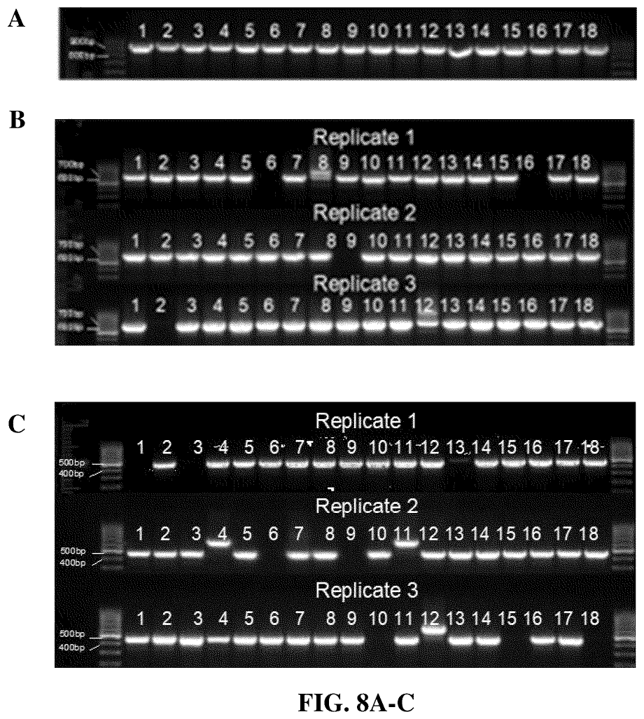

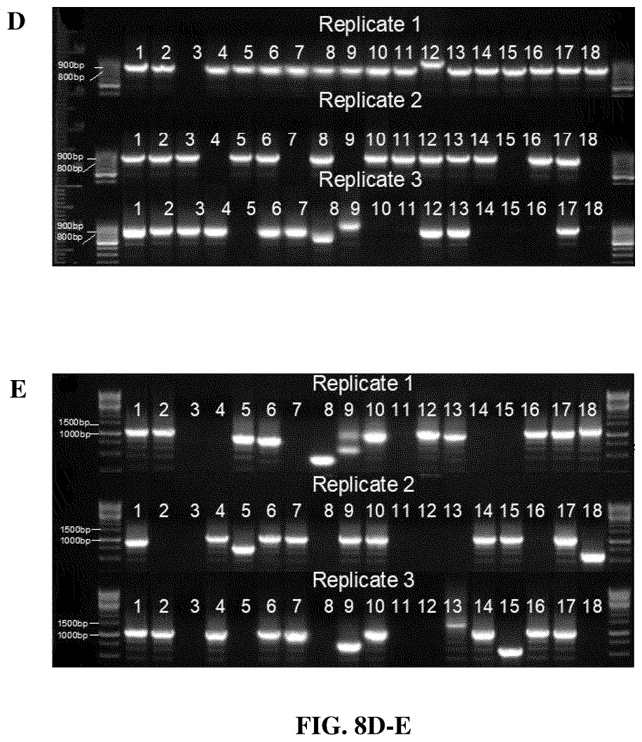

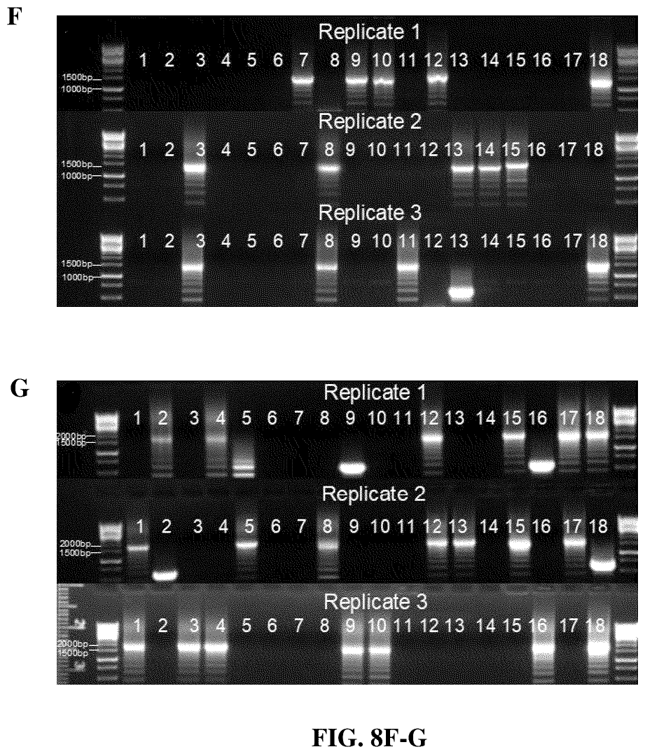

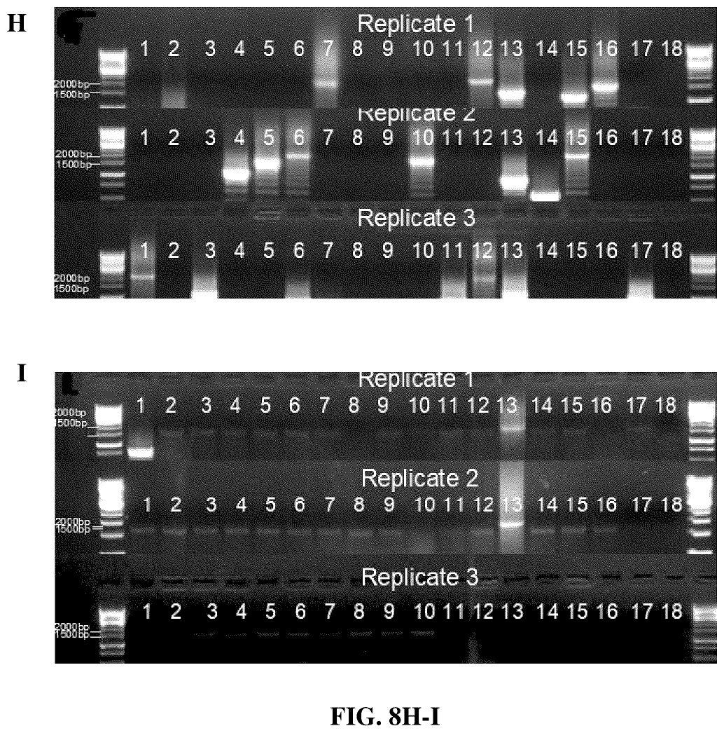

The PARAweb interface was created for steps 1 and 2, including the name, featured figure, drop-down menus, and upload zone. When the defined gRNA sequences are given, to select high-fidelity overhang sets, the step 3 is global optimization of overhangs (OHs) from gRNA sequences via 1) identification of candidate OHs from each of the 20-nt gRNA sequence; 2) identification of all overhang combinations with pairwise cross-match score<30 from identified candidate OHs in step 1); and 3) identification of the best overhang combination with the highest total self-match score for assembling the gRNA array, as illustrated in FIG. 4. The cross-match score and self-match score were used based on comprehensive profiling of four base overhang ligation fidelity by T4 DNA ligase.

Once the overhang is selected for each gRNA sequence, required oligos/primers are then generated in step 4. For each primer, the 5′ end of a template-specific sequence is orderly flanked by one BsaI restriction site, one specific 4-bp overhang sequence and one gRNA sequence. In step 5, each component DNA fragment is generated by combining corresponding Forward primer (F[n]), predefined template sequence and Reverse primer (R [n]). In step 6, assembled gRNA array sequence is generated by combining individual component DNA fragments from step 5. In step 7, assembled vector sequences is generated by connecting the user provided destination vector and assembled gRNA array sequence from step 6. In step 8, all above outputs, including required oligos (step 4), component DNA fragments (step 5), assembled gRNA array sequence (step 6), and assembled vector sequences (step 7), can be downloaded as individual text files.

In general, it is difficult to directly synthesize gRNA arrays due to their highly repetitive elements. Inspired by the multiplexed genome editing with the endogenous tRNA-processing system in rice, the PCR-based PARA method was developed to assemble tRNA-gRNA arrays using Golden Gate (GG) assembly (FIG. 1A). For orderly assembly of multiple fragments simultaneously, the fragment-specific sequences of overhangs (OHs) are an essential prerequisite. The fragment specific sequences of OH are determined by the restriction enzyme used to create the OH. In some embodiments, the OHs are two base OHs. In some embodiments, the OHs are three base OHs. In some embodiments, the OHs are four base OHs. In some embodiments, the OHs are five base OHs. In some embodiments, the OHs are six base OHs. In some embodiments, the OHs are seven base OHs. In some embodiments, the OHs are eight base OHs.

Unlike the modular cloning with predefined overhangs, in the PARA method the 4-bp overhangs are selected from distinct gRNA sequences and therefore, no scar sequences are introduced during cloning. Thus, the gRNA arrays can be divided into multiple individual DNA parts. Each of the DNA parts can be generated through PCR amplification of a predesigned template vector (FIG. 1A). [n+1] fragments can be used for the assembly of [n] gRNAs. Next, the DNA fragments are orderly ligated into a destination vector containing two predesigned BsaI restriction sites to form a gRNA array within an expression vector (FIG. 1A).

In some embodiments, proper design of the oligos (i.e., primers) required for PCR amplification of component fragments is an important step in the disclosed method. For each primer, the 5′ end of a template-specific sequence is orderly flanked by one BsaI restriction site, one specific 4-bp overhang sequence and one gRNA sequence (FIG. 1C). Two overhangs in the first forward primer and last reverse primer must be complementary with the sticky end of the destination vector digested by BsaI. Moreover, the selected overhangs must be distinctive, with a low similarity to each other to ensure the orderly assembly of gRNA arrays.

Using the disclosed PARA method, the expression vectors disclosed herein containing a gRNA array can be constructed within three days. As of this disclosure, a three day construction is the fastest way for assembly of gRNA arrays (FIG. 1D), saving up to 70% of time and efforts in comparison with traditional methods. Depending on the user preference and project requirements, the component DNA fragments can also be generated using commercial DNA synthesis or via annealing long oligos, allowing for high-throughput library synthesis.

### EXAMPLES

The following examples are set forth as being representative of the present disclosure. These examples are not to be construed as limiting the scope of the present disclosure as these and other equivalent embodiments will be apparent in view of the present disclosure, figures and accompanying claims.

### Example 1

To explore the capacity of PARA method, multi-gRNA assembly was performed with various number of gRNAs using the plant tRNA-gRNA system. Four target genes of Populus deltoides WV94 were selected from Phytozome and five gRNAs were designed for each gene using a gRNA design webtool, CHOPCHOP. Required oligonucleotides were designed manually as illustrated in FIG. 1C. The component fragments were generated through PCR amplification of the predesigned template vector type 1 followed by gel purification. Then, all component fragments were assembled into a modified pKSE401 vector followed by transformation in day one. Numerous colonies were observed on the selection medium in day two (FIG. 7).

Next, the colonies were analyzed by colony PCR (FIG. 8) and Sanger sequencing. As expected, the efficiency of GG assembly was gradually decreasing with the increase in total number of gRNAs (FIG. 4A). In two-gRNA assembly, target bands were observed in all selected colonies (n=18) (FIG. 8). When the number of gRNAs exceeds two, false positive colonies were detected on the selection medium. Interestingly, in four-gRNA assembly, 90% of the transformants harbored correctly assembled constructs (FIG. 2A). In the assembly between 6-10 gRNAs, the positive rate of transformants ranges from ˜ 50% to ˜80%. To explore the potential of PARA method, we further studied the assembly of gRNA arrays with up to 20 gRNAs. Over 25% of the analyzed transformants contained the correctly assembled constructs when the number of gRNA is under 16, while the positive rate decreased to below 10% when the number of gRNA is 16 and more (FIG. 2A). Two transformants were randomly selected from each replicate and ordered and oriented of the constructs were verified using Sanger sequencing. Overall, the data demonstrate that the PARA method is an effective approach for the one-pot assembly of gRNA arrays with up to 16 gRNAs.

### Example 2

In addition to the tRNA-gRNA system, polycistronic transcripts can also be processed post-transcriptionally into individual gRNAs by other RNA-cleaving enzymes, such as the CRISPR-associated RNA endoribonuclease Csy4 and ribozymes (RB). Recently, multiplexed CRISPR/Cas9 genome editing have been successfully applied in yeast, human cells, and plants. The PARA method was tested for the assembly of gRNA arrays based on Csy4 and ribozyme expression systems and compared the cloning efficiency of gRNA arrays containing the same set of eight gRNAs in different gRNA expression systems based on tRNA, Csy4, and ribozyme (FIGS. 2B and 2C). All component DNA fragments were generated by either PCR amplification of the predesigned template vector type 1 or annealing oligonucleotides. High-efficiency cloning, 80.0%, 73.4% and 63.0% was achieved in the Csy4 system, tRNA system and ribozyme system, respectively (FIG. 2C).

Recently, it was reported that multiplexed CRISPR/Cas12a was able to target multiple sites with high biallelic editing efficiency in rice using the processing system of the hammer head (HH) and hepatitis delta virus (HDV) ribozymes (FIG. 2B). However, the assembly of such a sophisticated construct is difficult and time-consuming, gRNA arrays were created comprising the same components in a one-step effort using the disclosed PARA method. Required oligonucleotides were designed manually using the same strategy as shown in FIG. 1B. Component fragments were generated through PCR amplification using a predesigned template vector type 2 (FIG. 6B). In the eight-gRNA assembly, approximately 36.43% of the analyzed transformants contained the correctly assembled construct (FIG. 2C). Other than PCR amplicons, the HH-HDV-RB array with eight gRNAs was also assembled successfully using synthesized DNA fragments (FIG. 7E). Altogether, the data demonstrate that the PARA method is a potent and robust approach for assembling gRNA arrays with different expression systems.

### Example 3

Multiple tRNA systems with organism-specific tRNA sequences have been used in plants, yeast, and drosophila. The Csy4 system has been used in plants, yeast, and human cells. The ribozyme and HH-HDV-RB systems have been used in plants. To simplify vector design and construction, the disclosed webtool, PARAweb, allows users to accurately design and simulate complex cloning procedures involving numerous gRNAs. The PARAweb tool is suitable for the design of all above-mentioned gRNA array expression systems (i.e., tRNA, Csy4, and Ribozyme for Cas9 as well as HH-HDV-RB for Cas12a) (FIG. 2D). Moreover, this web-based gRNA array tool is useful for the application of multiplexed CRISPR knockout, base editing, CRISPRa and CRISPRi in a wide range of organisms including animals, plants, and microbes. With given input gRNA sequences, PARAweb can generate PCR primers, component fragments, and linear assembled gRNA array sequence (FIG. 2E). When a valid destination vector sequence is given, PARAweb can also generate the assembled vector sequences containing the gRNA array (FIG. 5). Notably, the ligation frequency for each overhang pair in assembly reactions with BsaI-HFv2 and T4 DNA ligase, is utilized as a basic rule to select high-fidelity overhang sets in PARAweb. Eight poplar gRNAs used above were selected to test PARAweb, generating PCR primers, component fragments, linear assembled gRNA array sequence and assembled vector sequences. These component fragments were successfully PCR-amplified with the primers and ligated through linear ligation or cloned into a modified pKSE401 vector in SnapGene. Following the procedures described in FIG. 1, 55.6% positive colonies were detected in three biological replicates (FIG. 7F).

### Example 4: General Methods

### PCR Based Cloning

The component fragments were PCR-amplified using Q5® High-Fidelity 2X Master Mix (NEB #M0492L) with 65° C. annealing temperature.

### Colony PCR

Colony PCR was performed using GoTaq® Master Mixes (Promega) with 55° C. annealing temperature.

### Gel Purification

The PCR products were purified using Zymoclean Gel DNA Recovery Kit (ZYMO RESEARCH).

### Golden Gate Assembly

Assembly Reactions were performed in a thermocycler using BsaI-HFv2 (NEB #R3733) with suggested assembly protocol.

### Plasmid Sequencing

The plasmids were sanger sequenced using SimpleSeq Kit Premixed (Eurofins Genomics). The sequencing data were aligned with plasmid sequence in SnapGene.

E. coli Transformation

The transformation was performed using NEB® 5-alpha Competent E. coli (NEB #C2987H) following the manual.

### Plasmid Isolation

The plasmid DNA purification was performed using GenElute™ Plasmid Miniprep Kit (Sigma-Aldrich, PLN350-1KT).

### Oligos Annealing

Add the 2 oligo strands together in equal molar amounts. Heat the mixed oligonucleotides to 94° C. for 2 minutes and gradually cool.

### Vector Cloning

The U6 promoter in pKSE401 vector was replaced by a U3 promoter using HIFI DNA assembly and a window sequence was inserted between U3 promoter and its terminator. The template vectors were generated by inserting two gBlocks™ Gene Fragments (IDT) into modified pKSE401 vector via HIFI DNA assembly. Information for all primers and gBlocks used in this study can be found in Supplementary Data 1.

### Webtool Design

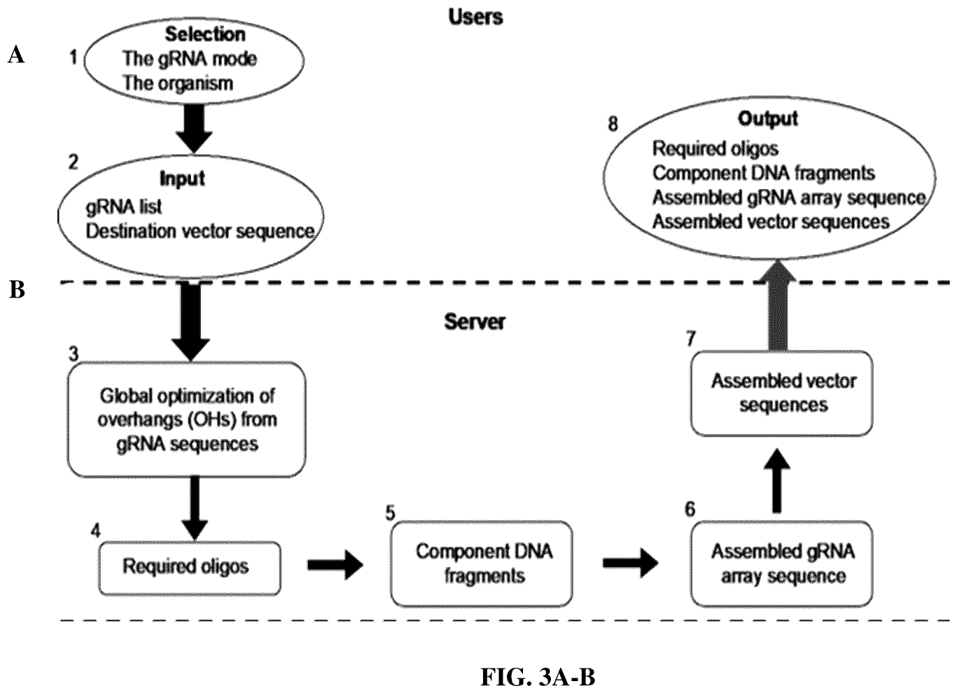

PARAweb is a web-tool that provides a complete workflow for the design and assembly of gRNA arrays for multiplex genome editing. The PARA webtool is built using standard html, CSS, and JavaScript components. The PARAweb tool features a series of drop-down menus that the user may interact with to choose the parameters for the design tool. Parameters include the type of multi-gRNA expression system, the ligation action, the appropriate restriction enzyme, and the organism type. Following parameter selection, the user drops a file containing the gRNA sequences of the gRNA array. The overhangs are chosen via algorithm (see below), and a list of primers is displayed in tabular color-coded format for PCR amplification of DNA fragments. When the complete sequences are downloaded, DNA constants relevant to specific gRNA mode set are used. The resulting text files contain the primers, the component DNA fragments of gRNA, and the complete gRNA array assembly sequence. FIG. 3 illustrates the workflow implemented in PARAweb.

1. A polycistronic guide RNA (gRNA) array, comprising:
nucleotide sequences of a plurality of guide RNAs (gRNAs) for use in a CRISPR-Cas system; wherein:
a) each nucleotide sequence of a gRNA in the array:
i) comprises a gRNA targeting sequence and a gRNA binding sequence, wherein the gRNA targeting sequence in each nucleotide sequence of a gRNA is unique to that gRNA; and the gRNA binding sequence is common to all the gRNAs in the array; and
ii) is linked at the 5′ end to a common RNA cleavage recognition sequence (5′ RCRS); and
b) upon cleavage by an RNA cleaving agent specific for the RNA cleavage recognition sequence, the polycistronic gRNA array generates the plurality of gRNAs.

2. The polycistronic gRNA array of claim 1, wherein each nucleotide sequence of a gRNA in the array is also linked at the 3′ end to a common RNA cleavage recognition sequence (3′ RCRS), wherein the 3′ RCRS is different from the 5′ RCRS.

3. The polycistronic gRNA array of claim 1, wherein the CRISPR-Cas system is a CRISPR-Cas9 system or a CRISPR-Cas12 system.

4. The polycistronic gRNA array of claim 1, wherein the 5′ RCRS and the 3′ RCRS are selected from a recognition sequence of a ribozyme, a recognition sequence of a tRNA ribonuclease, or a recognition sequence of a Csy4.

5. The polycistronic gRNA array of claim 4, wherein the recognition sequence of a ribozyme is the recognition sequence of Hammerhead ribozyme (HH) or the recognition sequence of hepatitis delta virus ribozyme (HDV).

6. The polycistronic gRNA array of claim 5, wherein one of the 5′ RCRS and the 3′ RCRS is the recognition sequence of HH, and the other one is the recognition sequence of HDV.

7. The polycistronic gRNA array of claim 4, wherein the tRNA ribonucleases are RNase P and RNase Z.

8. The polycistronic gRNA array of claim 1, wherein the CRISPR-Cas system is a CRISPR-Cas9 system, and wherein the 5′ RCRS is selected from a recognition sequence of a ribozyme, a recognition sequence of a tRNA ribonuclease, or a recognition sequence of Csy4.

9. The polycistronic gRNA array of claim 1, wherein the CRISPR-Cas system is a CRISPR-Cas12 system, wherein the nucleotide sequence of each gRNA comprises a LbCpf1 (Cas12a) CRISPR-RNA (crRNA) repeat at the 5′ end, wherein the crRNA repeat is downstream of the 5′ RCRS and upstream of the gRNA targeting sequence; and wherein the crRNA repeat in each nucleotide sequence of a gRNA is common to all the gRNAs in the array.

10. The polycistronic gRNA of claim 9, wherein the 5′ RCRS is the recognition sequence of a first ribozyme; and the 3′ end of the gRNA targeting sequence in each gRNA is linked to a common RCRS (3′ RCRS), wherein the 3′ RCRS comprises the recognition sequence of a second ribozyme; wherein the first and second ribozymes are not the same ribozyme; and wherein upon cleavage by the first and second ribozymes, the polycistronic gRNA array generates the plurality of individual gRNAs.

11. The polycistronic gRNA of claim 10, wherein:
a) the first ribozyme is Hammerhead ribozyme (HH), and the second ribozyme is hepatitis delta virus ribozyme (HDV); or
b) the first ribozyme is HDV and the second ribozyme is HH.

12. The polycistronic gRNA array of claim 1, wherein the array includes at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 gRNAs.

13. The polycistronic gRNA array of claim 1, wherein the array includes no more than 20 gRNAs.

14. A DNA encoding the polycistronic gRNA of claim 1.

15. A multiplex CRISPR vector, comprising:
a) the DNA of claim 14, and
b) a destination vector which comprises, from 5′ to 3′:
i) a promoter;
ii) a first recognition sequence of a type IIS restriction enzyme;
iii) the reverse complement of a second recognition sequence of the type IIS restriction enzyme, and
iv) a terminator;
wherein the DNA is integrated into the destination vector between the first recognition sequence and the reverse complement of the second recognition sequence of the type IIS restriction enzyme.

16. The multiplex CRISPR vector of claim 15, wherein the destination vector further comprises a pol II promoter, a Cas9 sequence and corresponding terminator.

17. The multiplex CRISPR vector of claim 15, wherein the destination vector further comprises a pol II promoter, a Cas12a sequence and corresponding terminator.

18. The multiplex CRISPR vector of claim 15, wherein the destination vector further comprises a marker sequence.

19. The multiplex CRISPR vector of claim 15, wherein the type IIS restriction enzyme is selected from the group consisting of BsaI, AarI, BbsI, BbsI-HF, BsmbI-v2, BspQI, BtgZI, Esp3I, PaqCI and SapI.

20. A plurality of component DNA fragments for assembly into a DNA encoding a polycistronic gRNA array, wherein:
the total number of component DNA fragments is n+1, the total number of gRNAs in the polycistronic gRNA array is n, and n is equal or greater than 2;
the component DNA fragments are designated as the first to the (n+1)^{th} component DNA fragments, and the gRNAs in the polycistronic gRNA array are designated as the first to the n^{th} gRNA;
the DNA encoding a polycistronic gRNA array is generated when the component DNA fragments are assembled in the order of the first to the (n+1)^{th} component DNA fragment in the 5′ to 3′ orientation, wherein:
a) the first component DNA fragment comprises, from 5′ to 3′:
i) the recognition sequence of a type IIS restriction enzyme,
ii) an upstream vector matching overhang sequence of variable length (e.g., 2, 3, 4, 5, or 6-bp),
iii) a nucleotide sequence encoding a common 5′ RNA cleavage recognition sequence (5′ RCRS),
iv) a nucleotide sequence encoding a 5′ portion of the targeting sequence of a first gRNA and comprising a downstream overhang sequence unique to the first gRNA; and
v) the reverse complement of the recognition sequence of the type IIS restriction enzyme;
b) for each of the second to the (n+1)^{th} component DNA fragments, with “p” representing a number from 2 to n, a p^{th} component DNA fragment comprises, from 5′ to 3′:
i) the recognition sequence of the type IIS restriction enzyme,
ii) a nucleotide sequence comprising an upstream overhang sequence and encoding a 3′ portion of the targeting sequence of the (p−1)^{th} gRNA, wherein the upstream overhang sequence is unique to the (p−1)^{th} gRNA and complementary to the downstream (e.g., 2, 3, 4, 5, or 6-bp) overhang sequence in the (p−1)^{th} component DNA fragment;
iii) a nucleotide sequence encoding a common gRNA binding sequence;
iv) a nucleotide sequence encoding the common 5′ RCRS,
v) a nucleotide sequence encoding a 5′ portion of the targeting sequence of the p^{th} gRNA and comprising a downstream overhang sequence unique to the p^{th} gRNA; and
vi) the reverse complement of the recognition sequence of the type IIS restriction enzyme; and
c) the (n+1)^{th} component DNA fragment comprises, from 5′ to 3′:
i) the recognition sequence of the type IIS restriction enzyme;
ii) a nucleotide sequence comprising an upstream overhang sequence and encoding a 3′ portion of the targeting sequence of the nth gRNA, wherein the upstream overhang sequence is unique to the nth gRNA and complementary to the downstream overhang sequence in the nth component DNA fragment;
iii) a nucleotide sequence encoding the common gRNA binding sequence;
iv) a downstream vector matching nucleotide overhang sequence; and
v) the reverse complement of the recognition sequence of the type IIS restriction enzyme.

21. The plurality of component DNA fragments of claim 20, wherein the type IIS restriction enzyme is selected from the group consisting of BsaI, AarI, BbsI, BbsI-HF, BsmbI-v2, BspQI, BtgZI, Esp3I, PaqCI and SapI.

22. A plurality of primer pairs for making a plurality of component DNA fragments to be assembled into a DNA encoding a polycistronic gRNA array, wherein:
the total number of primer pairs is n+1, for making n+1 component DNA fragments to be assembled into a DNA encoding a polycistronic gRNA array for n gRNAs, with n being equal or greater than 2;
the primer pairs are designated as the first to the (n+1)^{th} primer pair, the component DNA fragments are designated as the first to the (n+1)^{th} component DNA fragments, and the gRNAs in the polycistronic gRNA array are designated as the first to the nth gRNA;
the DNA encoding a polycistronic gRNA array is generated when the component DNA fragments are assembled in the order of the first to the (n+1)^{th} component DNA fragment in the 5′ to 3′ orientation, wherein:
a) the first primer pair comprises a forward primer and a reverse primer, wherein:
the forward primer of the first primer pair comprises, from 5′ to 3′:
(i) the recognition sequence of a type IIS restriction enzyme;
(ii) an upstream vector matching overhang sequence; and
(iii) a template specific sequence (e.g., a sequence encoding a 5′ portion of an RNA cleavage recognition sequence (RCRS); and
the reverse primer of the first primer pair comprises, from 5′ to 3′:
(i) the recognition sequence of the type IIS restriction enzyme;
(ii) a sequence encoding a 5′ portion of the targeting sequence of the first gRNA and comprising a downstream overhang sequence unique to the first gRNA; and
(iii) a template specific sequence (e.g., a sequence encoding a 3′ portion of the RNA cleavage recognition sequence (RCRS);
b) for each of the second to the (n+1)^{th} primer pairs, with “p” representing a number from 2 to n, a p^{th} primer pair comprises a forward primer and a reverse primer, wherein:
the forward primer of the p^{th} primer pair comprises, from 5′ to 3′:
(i) the recognition sequence of the type IIS restriction enzyme,
(ii) a nucleotide sequence comprising an upstream overhang sequence and encoding a 3′ portion of the targeting sequence of the (p−1)^{th} gRNA, wherein the upstream overhang sequence is unique to the (p−1)^{th} gRNA and complementary to the downstream overhang sequence in the reverse primer of the (p−1)^{th} primer pair, and
(iii) a template specific sequence (e.g., a sequence encoding a 5′ portion of a common gRNA binding sequence); and
the reverse primer of the p^{th} primer pair comprises, from 5′ to 3′:
(i) the recognition sequence of the type IIS restriction enzyme;
(ii) a sequence encoding a 5′ portion of the targeting sequence of the p^{th} gRNA and comprising a downstream overhang sequence unique to the p^{th} gRNA; and
(iii) a template specific sequence (e.g., a sequence encoding a 3′ portion of an RCRS);
c) the (n+1)^{th} primer pair comprises a forward primer and a reverse primer, wherein:
the forward primer the (n+1)^{th} primer pair comprises, from 5′ to 3′:
(i) the recognition sequence of the type IIS restriction enzyme;
(ii) a nucleotide sequence comprising an upstream overhang sequence and encoding a 3′ portion of the targeting sequence of the n^{th} gRNA, wherein the upstream overhang sequence is unique to the n^{th} gRNA and complementary to the downstream (4-bp) overhang sequence in the reverse primer of the n^{th} primer pair; and
(iii) a template specific sequence (e.g., a sequence encoding a 5′ portion of the common gRNA binding sequence); and
the reverse primer of the (n+1)^{th} primer pair comprises, from 5′ to 3′;
(i) the recognition sequence of the type IIS restriction enzyme;
(ii) a downstream vector matching overhang sequence; and
(iii) a template specific sequence (e.g., a sequence encoding a 3′ portion of the common gRNA binding sequence).

23. The plurality of primer pairs of claim 22, further wherein the length of the overhang sequences range from 2 nucleotides to 8 nucleotides based on the type IIS restriction enzyme.

24. The plurality of primer pairs of claim 22, wherein the type IIS restriction enzyme recognition site is flanked on the 5′ end by two additional base pairs for enhancing the restriction enzyme digestion of Polymerase Chain Reaction products.

25. The plurality of primer pairs of claim 22, wherein the type IIS restriction enzyme is BsaI and the overhangs are 4 nucleotides in length.

26. A method of making the multiplex CRISPR vector of claim 15, the method comprising:
a) selecting an organism and gRNA mode;
b) inputting a gRNA list and destination vector sequences into a database; wherein the gRNA list comprises nucleotide sequences of a plurality of gRNAs, wherein each nucleotide sequence of a gRNA in the array comprises a gRNA targeting sequence and a gRNA binding sequence;
c) optimizing nucleotide overhangs from gRNA sequences, comprising;
i) identifying candidate overhangs from each of the gRNA sequences;
ii) identifying all overhang combinations with a pairwise crossmatch score of less than 30 from identified candidate overhangs in step (c)(i); and
iii) identifying the best overhang combination with the highest total self-match score for assembling the gRNA array;
d) designing primer pairs;
e) generating component DNA fragments by combining the corresponding forward primer (F[n]), predefined template sequence, and reverse primer PCR amplification, wherein:
i) n+1 component DNA fragments are to be assembled into a DNA encoding a polycistronic gRNA array for n gRNAs, with n being equal or greater than 2;
ii) the component DNA fragments are designated as the first to the (n+1)^{th} component DNA fragments, and the gRNAs in the polycistronic gRNA array are designated as the first to the n^{th} gRNA; and
iii) the DNA encoding a polycistronic gRNA array is generated when the component DNA fragments are assembled in the order of the first to the (n+1)^{th} component DNA fragment in the 5′ to 3′ orientation;
f) assembling the gRNA array sequence by combining individual component DNA fragments from step (e); and
g) generating assembled vector sequences by connecting user-provided destination vector and assembled gRNA array sequence from step (f).

27.-35. (canceled)


SEQUENCE LISTING (10 sequences)

SEQ ID NO: 1 - DNA, 27 bp, synthetic construct
  misc_feature 1..27: note = Forward Primer FP[1]
  source 1..27: mol_type = other DNA
taggtctccggtcaacaaagcaccagt

SEQ ID NO: 2 - DNA, 35 bp, synthetic construct
  misc_feature 1..35: note = Reverse Primer RP[1]
  misc_feature 10..17: note = n is a, c, g, or t
  source 1..35: mol_type = other DNA
atggtctcannnnnnnntgcaccagccgggaatcg

SEQ ID NO: 3 - DNA, 40 bp, synthetic construct
  misc_feature 1..40: note = Forward Primer FP[n]
  misc_feature 10..17: note = n is a, c, g, or t
  source 1..40: mol_type = other DNA
taggtctccnnnnnnnngttttagagctagaaatagcaag

SEQ ID NO: 4 - DNA, 34 bp, synthetic construct
  misc_feature 1..34: note = Reverse Primer RP [n]
  misc_feature 10..17: note = n is a, c, g, or t
  source 1..34: mol_type = other DNA
atggtctcannnnnnnntgcaccagccggaatcg

SEQ ID NO: 5 - DNA, 40 bp, synthetic construct; identical to SEQ ID NO: 3
  misc_feature 1..40: note = Forward Primer FP[n+1]
  misc_feature 10..17: note = n is a, c, g, or t
  source 1..40: mol_type = other DNA

SEQ ID NO: 6 - DNA, 32 bp, synthetic construct
  misc_feature 1..32: note = Reverse Primer RP[n+1]
  source 1..32: mol_type = other DNA
atggtctcaccactttttcaagttgataacgg

SEQ ID NO: 7 - DNA, 69 bp, synthetic construct
  misc_feature 1..69: note = Primer-WS-Terminator
  source 1..69: mol_type = other DNA
agggagcaccattggtcggagaccaacggtctcggtggcaccgagtcggtgcttttttttccctttcct

SEQ ID NO: 8 - DNA, 69 bp, synthetic construct
  misc_feature 1..69: note = Primer-WS-Terminator
  source 1..69: mol_type = other DNA
tccctcgtggtaaccagcctctggrrgccagagccaccgtggctcagccacgaaaaaaaagggaaagga

SEQ ID NO: 9 - DNA, 46 bp, synthetic construct
  misc_feature 1..46: note = Window Sequence
  source 1..46: mol_type = other DNA
ggtcggagaccaacggtctcggtggcaccgagtcggtgcttttttt

SEQ ID NO: 10 - DNA, 46 bp, synthetic construct
  misc_feature 1..46: note = Window Sequence
  source 1..46: mol_type = other DNA
ccagcctctggrrgccagagccaccgtggctcagccacgaaaaaaa